(19) United States
(12) Patent Application Publication
(10) Pub. No.: US 2024/0140947 A1
(43) Pub. Date: May 2, 2024

(54) COMPOUND FOR THE TREATMENT OF CANCER

(71) Applicant: Pfizer Inc., New York, NY (US)

(72) Inventors: Fengjuan Cao, San Diego, CA (US); Kevin Daniel Freeman-Cook, Carlsbad, CA (US); Rhys Marsden Jones, Carlsbad, CA (US); Scott Channing Sutton, San Diego, CA (US)

(73) Assignee: Pfizer Inc., New York, NY (US)

(21) Appl. No.: 18/488,191

(22) Filed: Oct. 17, 2023

Related U.S. Application Data
(60) Provisional application No. 63/495,836, filed on Apr. 13, 2023.
(60) Provisional application No. 63/379,924, filed on Oct. 18, 2022.

(51) Int. Cl. C07D 471/04; A61K 45/06; A61P 35/00

(52) U.S. Cl. CPC C07D 471/04; A61K 45/06; A61P 35/00

(57) ABSTRACT

The invention relates to 4-((6-(2,2-Difluoroethyl)-8-(2-hydroxy-2-methylcyclopentyl)-7-oxo-7,8-dihydropyrido[2,3-d]pyrimidin-2-yl)amino)piperidine-1-sulfonamide or pharmaceutically acceptable salts thereof, to compositions containing them, to processes for their preparation, to intermediates used in such processes, and to methods of using such compounds, salts and compositions for the treatment of abnormal cell growth, including cancer. The disclosure also relates to its crystalline form 1, to pharmaceutical compositions comprising form 1, and to the use of form 1 for the treatment of cancers.

### FIELD

The present disclosure relates to novel 4-((6-(2,2-difluoroethyl)-8-(2-hydroxy-2-methylcyclopentyl)-7-oxo-7,8-dihydropyrido[2,3-d]pyrimidin-2-yl)amino)piperidine-1-sulfonamide of Formula (I) and its pharmaceutically acceptable salts, to pharmaceutical compositions comprising such compounds and salts, and to the uses thereof for the treatment of CDKs related diseases such as cancers. The present disclosure also relates to a crystalline form of 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide free base (herein as “Form 1”), to pharmaceutical compositions comprising Form 1, and to the use of Form 1 for the treatment of CDKs related diseases such as cancers.

### BACKGROUND

Overcoming checkpoints that impede advancement through the cell cycle is fundamentally required for tumors to advance. The cell cycle consists of the mitotic phase where DNA segregation and cellular fission are completed and an interphase where the G1 and G2 checkpoints occur before and after DNA synthesis, respectively (Choi et al., Signaling through cyclin D-dependent kinases. Oncogene 2014, 33(15):1890-903). Cells in G1 require the activity of cyclin dependent kinase (CDK)4/6-cyclin (CCN)-D to phosphorylate the retinoblastoma (Rb) tumor suppressor which is further phosphorylated by CDK2 CCNE. Phosphorylation of Rb results in its release from pro-transcriptional-complexes containing the E2F transcription factor (TF) that regulate expression of genes required for S phase completion. Multiple genetic lesions have been identified that augment this particular signaling-event in various tumor types. For instance, CCND1 and CCNE1 amplification are common, as are deletions of Rb or the endogenous CDK4-CCND inhibitor, p16. The prediction that pharmacological targeting of the CDK4/6 Rb axis would be efficacious in cancer, was borne out by the clinical success of palbociclib- in combination with anti-estrogens in hormone receptor (HR)+ breast cancer (Cristofanilli et al., Fulvestrant plus palbociclib versus fulvestrant plus placebo for treatment of hormone-receptor-positive, HER2-negative metastatic breast cancer that progressed on previous endocrine therapy (PALOMA-3): Final analysis of the multicentre, double-blind, phase 3 randomised controlled trial. Lancet Oncol 2016, 17(4):425-39).

Targeted therapies often result in initial clinical benefit followed by acquired resistance through mutation or activation of orthologous pathways (Chong et al., Nat. Med. 2013,19(11):1389-400). Resistance to palbociclib in nonclinical cellular models can occur via loss of Rb or upregulation of CCNE1, while CCNE1-amplified cell lines are sensitive to CDK2/4/6 inhibition (Herrera-Abreu, et al., Cancer Res. 2016, 76(8):2301-13). In addition to ER positive breast cancer patients refractory to palbociclib, several tumor types including triple negative breast cancer (TNBC), ovarian, and others, have Cyclin E amplified alleles (CCNE1 or CCNE2) (The Cancer Genome Atlas Network, Nature 2012, 490(7418):61-70).

Based on this rationale the development of a potent CDK2/4/6 inhibitor has the potential to be an effective therapy in the treatment of positive HER2 negative advanced or metastatic breast cancer and other tumor types with increased Cyclin E expression/CDK activity including TNBC and ovarian cancer. CDK6 inhibition is a known safety liability for consideration which can lead to hematologic adverse events in human which has been observed for a number of CDK4/6 inhibitors (Desnoyers, et al., Cancer Treat. Rev. 2020, 90:102086, PMID: 32861975; Sun et al., J. Clin. Pharm. 2017, 57(9):1159-1173; Goel, et al., Nat. Rev. Cancer 2022, 22:356-372). These adverse events (primarily neutropenia) can be dose limiting and potentially impact the achievable efficacy for these molecules. However, although too much CDK6 inhibition can lead to adverse events it remains a potentially important CDK to inhibit in addition to CDK2/4 as it is also linked to CDK6-driven resistance during prolonged use with CDK4/6 inhibitors (Yang, et al., Oncogene 2017, 36:2255-2264).

Accordingly, there remains a need for new CDK2/4/6 inhibitors with reduced incidence and severity of hematologic adverse events which may allow higher exposure and lead to more robust inhibition cell cycle inhibition and improved efficacy.

### SUMMARY

The present disclosure provides, in part, novel 4-((6-(2,2-difluoroethyl)-8-(2-hydroxy-2-methylcyclopentyl)-7-oxo-7,8-dihydropyrido[2,3-d]pyrimidin-2-yl)amino)piperidine-1-sulfonamide of Formula (I), and pharmaceutically acceptable salts thereof. The compounds of the present disclosure and pharmaceutically acceptable salts thereof can inhibit the activity of CDKs, including CDK2, CDK4 and/or CDK6, thereby effecting biological functions. Also provided are pharmaceutical compositions and medicaments, comprising the compound or salts of Formula (I), alone or in combination with additional anticancer therapeutic agents or palliative agents. The present disclosure also provides, in part, methods for preparing the compound or salts of Formula (I), composition comprising the compound or salts of Formula (I), and methods of using thereof.

According to an embodiment of the disclosure there is provided a compound of Formula (I)

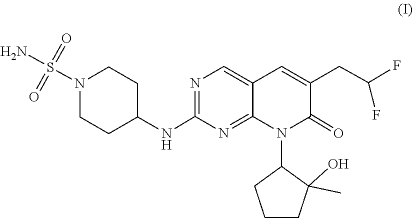 [SMILES: CC1(O)CCCC1n1c(=O)c(CC(F)F)cc2cnc(NC3CCN(S(N)(=O)=O)CC3)nc21]

or a pharmaceutically acceptable salt thereof.

In some aspects and embodiments, the disclosure provides an anhydrous crystalline form of 4-((6-(2,2-difluoroethyl)-8-((l R,2R)-2-hydroxy-2-methylcyclopentyl)-7-oxo-7,8-dihydropyrido[2,3-d]pyrimidin-2-yl)amino)piperidine-1 -sulfonamide free base (herein as “Form 1”)

Described below are embodiments of the disclosure, where for convenience Embodiment 1 (El) is identical to the embodiment of Formula (I) provided above.

It is to be understood that both the foregoing general description and the following detailed description are exemplary and explanatory only and are not restrictive of the disclosure, as claimed.

### BRIEF DESCRIPTION OF THE DRAWINGS

FIG. 1 shows the PXRD spectrum of anhydrous crystalline 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide free base (Form 1).

FIG. 2 shows histology images of mouse bone marrow biopsy from treatment Group 1 (75 mg/kg/dose BID Compound 1), Group 2 (150 mg/kg/dose BID PF-06873600) and vehicle control Group 3.

FIG. 3 shows a graph plotting plasma free (nM) over time (h) in the mouse exposures in toxicity studies in treatment Group 1 (75 mg/kg/dose BID Compound 1) on day 14 and Group 2 (150 mg/kg/dose BID PF-06873600) on day 8.

### DETAILED DESCRIPTION

The present disclosure may be understood more readily by reference to the following detailed description of the embodiments of the disclosure and the Examples included herein. It is to be understood that this disclosure is not limited to specific synthetic methods of making that may of course vary. It is to be also understood that the terminology used herein is for the purpose of describing specific embodiments only and is not intended to be limiting.
E1 A compound of Formula (I) or a pharmaceutically acceptable salt thereof, as shown above.
E2 The compound of embodiment E1, or a pharmaceutically acceptable salt thereof, wherein the compound of Formula (I) has the absolute stereochemistry as shown in Formula (I-A), (I-B), (I-C), or (I-D):

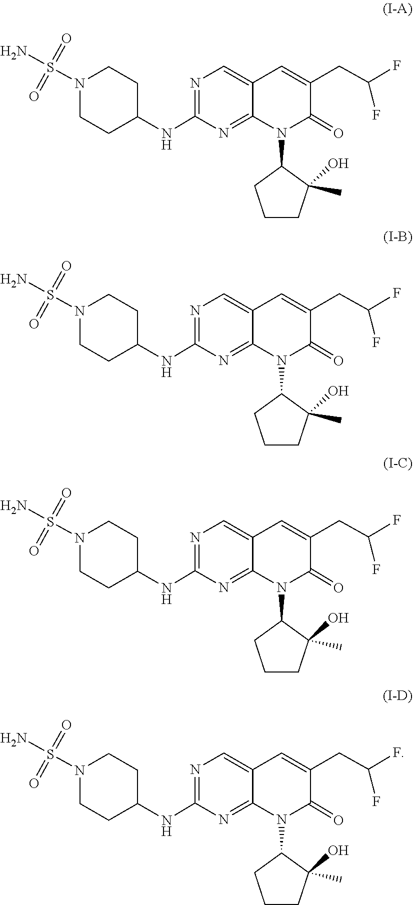 [SMILES: C[C@@]1(O)CCC[C@H]1n1c(=O)c(CC(F)F)cc2cnc(NC3CCN(S(N)(=O)=O)CC3)nc21 ; C[C@@]1(O)CCC[C@@H]1n1c(=O)c(CC(F)F)cc2cnc(NC3CCN(S(N)(=O)=O)CC3)nc21 ; C[C@]1(O)CCC[C@H]1n1c(=O)c(CC(F)F)cc2cnc(NC3CCN(S(N)(=O)=O)CC3)nc21 ; C[C@]1(O)CCC[C@@H]1n1c(=O)c(CC(F)F)cc2cnc(NC3CCN(S(N)(=O)=O)CC3)nc21]

E3 A compound which is 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-l-sulfonamide of Formula (I-A) or a pharmaceutically acceptable salt thereof.
E4 A compound which is 4-((6-(2,2-difluoroethyl)-8-((1S,2R)-2-hydroxy-2-methylcyclopentyl)-7-oxo-7,8-dihydropyrido[2,3-d]pyrimidin-2-yl)amino)piperidine-l-sulfonamide of Formula (I-B) or a pharmaceutically acceptable salt thereof.
E5 A compound which is 4-((6-(2,2-difluoroethyl)-8-((1R,2S)-2-hydroxy-2-methylcyclopentyl)-7-oxo-7,8-dihydropyrido[2,3-d]pyrimidin-2-yl)amino)piperidine-l-sulfonamide of Formula (I-C) or a pharmaceutically acceptable salt thereof.
E6 A compound which is 4-((6-(2,2-difluoroethyl)-8-((1S,2S)-2-hydroxy-2-methylcyclopentyl)-7-oxo-7,8-dihydropyrido[2,3-d]pyrimidin-2-yl)amino)piperidine-1-sulfonamide of Formula (I-D) or a pharmaceutically acceptable salt thereof.
E7 The compound according to any one of embodiments E1-E3 which is 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide.
E8 The compound according to any one of embodiments E1-E3 having the chemical structure

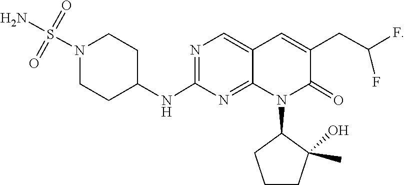 [SMILES: C[C@@]1(O)CCC[C@H]1n1c(=O)c(CC(F)F)cc2cnc(NC3CCN(S(N)(=O)=O)CC3)nc21]

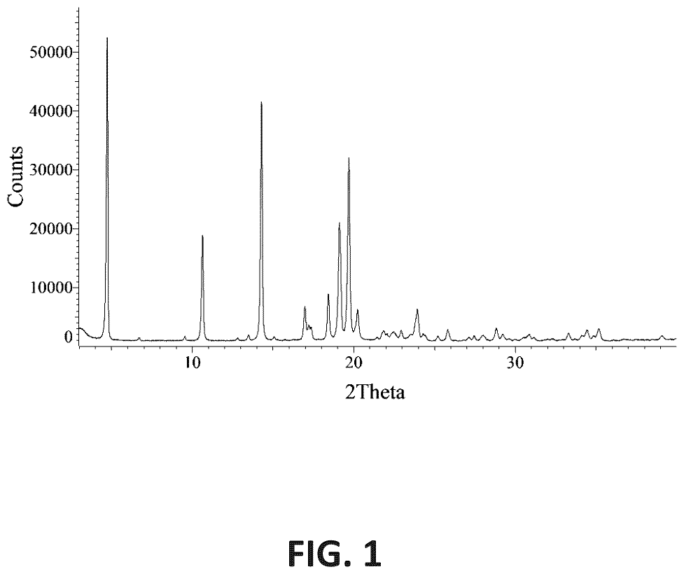

E9 A pharmaceutically acceptable salt of 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide.
E10 An anhydrous crystalline form of 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide, free base (Form 1) having a powder X-ray diffraction (PXRD) pattern comprising one, two, three, four, five, or more than five peaks in Table 1 in °2θ±0.2°2θ.
E11 An anhydrous crystalline form of 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide, free base (Form 1) having a powder X-ray diffraction (PXRD) pattern comprising peaks at 2θ values of: 4.8, 14.3 and 19.7 °2θ±0.2 °2θ.
E12 The anhydrous crystalline form according to any one of embodiments E10-E11, having a PXRD pattern further comprising a peak at a 2θ value of: 10.6 °2θ±0.2 °2θ.
E13 The anhydrous crystalline form according to any one of embodiments E10-E12, having a PXRD pattern further comprising a peak at a 2θ value of: 19.1 °2θ±0.2 °2θ.
E14 The anhydrous crystalline form according to any one of embodiments E10-E13, having a PXRD pattern comprising peaks at 2θ values essentially the same as shown in FIG. 1.
E15 The anhydrous crystalline form according to any one of embodiments E10-E14, wherein the crystalline form is substantially pure.
E16 A compound of Formula (II)

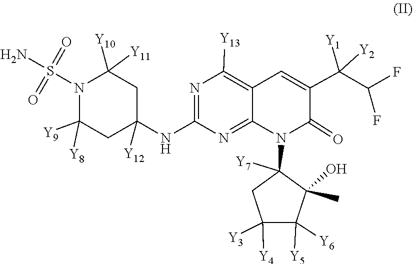

or a pharmaceutically acceptable salt thereof,
wherein each of Y₁, Y₂, Y₃, Y₄, Y₅, Y₆, Y₇, Y₈, Y₉, Y₁₀, Y₁₁, and Y₁₂ is independently halogen or deuterium.
E17 The compound of embodiment E16, wherein each of Y₈ and Y₉ is deuterium.
E18 The compound according to any one of embodiments E16-E17, wherein each of Y₁₀ and Y₁₁ is deuterium.
E19 The compound according to any one of embodiments E16-E18, wherein each of Y₁ and Y₂ is deuterium.
E20 The compound according to any one of embodiments E16-E19, wherein Y₁₂ is deuterium.
E21 The compound according to any one of embodiments E16-E20, wherein Y₇ is deuterium.
E22 The compound according to any one of embodiments E16-E21, wherein Y₁₃ is deuterium.
E23 The compound according to any one of embodiments E16-E22, wherein each of Y₃ and Y₄ is deuterium.
E24 The compound according to any one of embodiments E16-E23, wherein each of Y₅ and Y₆ is deuterium.
E25 A pharmaceutical composition comprising a compound according to any one of embodiments E1 to E24 or a pharmaceutically acceptable salt thereof, and at least one pharmaceutically acceptable excipient.
E26 A method for treating cancer, comprising administering to a subject in need thereof a therapeutically effective amount of the compound of any one of embodiments E1 to E25, or a pharmaceutically acceptable salt thereof.
E27 The method for treating cancer according to embodiment E26, further comprising administering an additional anticancer therapeutic agent.
E28 The method for treating cancer according to any one of embodiments E26 to E27, wherein the cancer is selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer and thyroid cancer.
E29 The method for treating cancer of embodiment E28, wherein the cancer is breast cancer or ovarian cancer.
E30 The method for treating cancer of embodiment E29, wherein the breast cancer or ovarian cancer is characterized by amplification or overexpression of cyclin E1 (CCNE1) or cyclin E2 (CCNE2).
E31 The method for treating cancer according to any one of embodiment E28 or E29, wherein the breast cancer is ER-positive/HR-positive breast cancer, HER2-negative breast cancer, ER-positive/HR-positive breast cancer, HER2-positive breast cancer, triple negative breast cancer (TNBC), or inflammatory breast cancer.
E32 The method of treating cancer according to any one of embodiments E28 to E31, wherein the breast cancer is advanced or metastatic breast cancer.
E33 A compound according to any one of embodiments El to E24 or a pharmaceutically acceptable salt, for use as a medicament.
E34 A compound according to any one of embodiments El to E24 or a pharmaceutically acceptable salt, for use in the treatment of cancer.
E35 Use of a compound according to any one of embodiments El to E24 or a pharmaceutically acceptable salt, for the manufacture of a medicament for the treatment of cancer.
E36 A pharmaceutical combination comprising a compound according to any one of embodiments E1 to E24 or a pharmaceutically acceptable salt, at least one additional therapeutic agent, and at least one pharmaceutically acceptable excipient.

Each of the embodiments described herein may be combined with any other embodiment(s) described herein not inconsistent with the embodiment(s) with which it is combined.

### Definitions

Unless otherwise defined herein, scientific, and technical terms used in connection with the present disclosure have the meanings that are commonly understood by those of ordinary skill in the art.

The disclosure described herein suitably may be practiced in the absence of any element(s) not specifically disclosed herein.

The present disclosure provides the compound of Formula (I), including stereoisomers of Formula (I) having the structures of Formula (I-A), (I-B), (I-C) and (I-D), and intermediates used in the preparation thereof, refer to as “compound of the disclosure.” One of ordinary skill in the art will appreciate that the compound of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that the compound of the disclosure include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

The disclosure described herein may be suitably practiced in the absence of any element(s) not specifically disclosed herein. Thus, for example, in each instance herein any of the terms “comprising”, “consisting essentially of”, and “consisting of” may be replaced with either of the other two terms.

As used herein, the term “essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (2θ) will show some variability, typically as much as ±0.2°. Further, one skilled in the art will appreciate that relative peak intensities will show inter-apparatus variability, as well as variability due to the degree of crystallinity, preferred orientation, prepared sample surface, and other factors known to those skilled in the art and should be taken as qualitative measures only.

As used herein, the term “crystalline” means having a regularly repeating arrangement of molecules or external face planes. Crystalline forms may differ with respect to thermodynamic stability, physical parameters, x-ray structure and preparation processes.

As used herein, the term “anhydrous” refers to a crystalline form that only contains the active pharmaceutical ingredient (API) as part of its crystalline lattice.

As used herein, the term “solvate” describes a molecular complex comprising a compound (e.g., the API of a drug product) and a stoichiometric or non-stoichiometric amount of one or more solvent molecules (e.g., water or ethanol). When the solvent is tightly bound to the compound, the resulting complex will have a well-defined stoichiometry that is independent of humidity. When, however, the solvent is weakly bound, as in channel solvates and hygroscopic compounds, the solvent content will be dependent on humidity and drying conditions. In such cases the complex will often be non-stoichiometric.

As used herein, the term “hydrate” describes a solvate comprising the compound and a stoichiometric or non-stoichiometric amount of water. A “monohydrate” is a hydrate comprising one molecule of water per molecule of compound (i.e., a 1:1 stoichiometry of water to compound).

As used herein, the term “substantially pure” means that the crystalline or amorphous form described as substantially pure comprises less than 5%, preferably less than 3%, and more preferably less than 1% by weight of impurities, including any other physical form of the compound. Alternatively, the crystalline or amorphous form described as substantially pure may be expressed as >95% pure, preferably >97% pure, and more preferably >99% pure, in each case by weight of impurities, including any other physical form of the compound.

As used herein, “endocrine therapy” means an aromatase inhibitor, a selective estrogen receptor degrader (SERD), or a selective estrogen receptor modulator (SERM). In certain embodiments, endocrine therapy includes fulvestrant, tamoxifen, toremifene, anastrozole, exemestane, or letrozole.

As used herein, the term “pharmaceutically acceptable” means the substance (e.g., the compounds described herein) and any salt thereof, or composition containing the substance or salt of the disclosure is suitable for administration to a subject or patient.

As used herein, a “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

As used herein, “excipient” as used herein describes any ingredient other than the compound(s) of the invention. The choice of excipient will to a large extent depend on factors such as the mode of administration, the effect of the excipient on solubility and stability, and the nature of the dosage form. The term “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

As used herein, the term “subject” refers to a human or animal subject. In certain preferred embodiments, the subject is a human.

As used herein, the term “abnormal cell growth” refers to cell growth that is independent of normal regulatory mechanisms (e.g., loss of contact inhibition). Abnormal cell growth may be benign (not cancerous), or malignant (cancerous). Abnormal cell growth includes the abnormal growth of: (1) tumor cells (tumors) that show increased expression of CDK2; (2) tumors that proliferate by aberrant CDK2 activation; (3) tumors characterized by amplification or overexpression of CCNE1 and/or CCNE2; and (4) tumors that are resistant to endocrine therapy, HER2 antagonists or CDK4/6 inhibition.

As used herein, the term “additional anticancer therapeutic agent” means any one or more therapeutic agent, other than a compound of the invention, that is or can be used in the treatment of cancer, such as agents derived from the following classes: mitotic inhibitors, alkylating agents, antimetabolites, antitumor antibiotics, topoisomerase I and II inhibitors, plant alkaloids, hormonal agents and antagonists, growth factor inhibitors, radiation, inhibitors of protein tyrosine kinases and/or serine/threonine kinases, cell cycle inhibitors, biological response modifiers, enzyme inhibitors, antisense oligonucleotides or oligonucleotide derivatives, cytotoxics, and immuno-oncology agents.

As used herein, the term “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

As used herein, the term “treating” means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

As used herein, the term “therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

### Salts

Salts encompassed within the term “pharmaceutically acceptable salts” refer to the compound of this disclosure which is generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula (I) may also include other salts of such compound which are not necessarily pharmaceutically acceptable salts, which may be useful as intermediates for one or more of the following: 1) preparing the compound of Formula (I); 2) purifying the compound of Formula (I); 3) separating enantiomers of the compound of Formula (I); or 4) separating diastereomers of the compounds of Formula (I).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of 4-((6-(2,2-Difluoroethyl)-8-(2-hydroxy-2-methylcyclopentyl)-7-oxo-7,8-dihydropyrido[2,3-d]pyrimidin-2-yl)amino)piperidine-1-sulfonamide may be prepared by methods well known to one skilled in the art, including but not limited to the following procedures
(i) by reacting 4-((6-(2,2-Difluoroethyl)-8-(2-hydroxy-2-methylcyclopentyl)-7-oxo-7,8-dihydropyrido[2,3-d]pyrimidin-2-yl)amino)piperidine-1-sulfonamide with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of 4-((6-(2,2-Difluoroethyl)-8-(2-hydroxy-2-methylcyclopentyl)-7-oxo-7,8-dihydropyrido[2,3-d]pyrimidin-2-yl)amino)piperidine-1-sulfonamide to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compound of the disclosure, and pharmaceutically acceptable salts thereof, may exist in unsolvated and solvated forms.

In addition, the compound of the disclosure may also include other solvates of such compounds which are not necessarily pharmaceutically acceptable solvates, which may be useful as intermediates for one or more of the following: 1) preparing the compound of the disclosure; 2) purifying the compound of the disclosure; 3) separating enantiomers of the compound of the disclosure; or 4) separating diastereomers of the compound of the disclosure.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the compound of the present disclosure and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17;1889-1896, by O. Almarsson and M. J. Zaworotko (2004). For a general review of multi-component complexes, see J Pharm Sci, 64(8), 1269-1288, by Haleblian (August 1975).

### Solid Form

The compound of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compound of the disclosure may also exist in a mesomorphic state (mesophase or liquid crystal) when subjected to suitable conditions. The mesomorphic state is intermediate between the true crystalline state and the true liquid state (either melt or solution) and consists of two-dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

In some embodiments, the present disclosure provides an anhydrous crystalline form of 4-((6-(2,2-difluoroethyl)-8-((1 R,2R)-2-hydroxy-2-methylcyclopentyl)-7-oxo-7,8-dihydropyrido[2,3-d]pyrimidin-2-yl)amino)piperidine-1-sulfonamide of Formula (I-A), free base (“Form 1”). In some embodiments, Form 1 which is substantially pure and free of alternative forms.

In some embodiments, Form 1 was characterized by Powder X-Ray Diffraction (PXRD). Such crystalline forms may be further characterized by additional techniques, such as Raman spectroscopy, and ¹³C and ¹⁹F solid state NMR spectroscopy, Fourier-Transform InfraRed Spectroscopy (FTIR), Differential Scanning calorimetry (DSC), Thermogravimetric Analysis (TGA) or Differential Thermal Analysis (DTA).

In some embodiments, the disclosure provides Form 1 which is characterized by having a PXRD pattern (2θ) comprising: (a) one, two, three, four, five, or more than five peaks selected from the group consisting of the peaks in Table 1 in °2θ±0.2 °2θ; (b) one, two, three, four or five peaks selected from the group consisting of the characteristic peaks in Table 1 in °2θ±0.2 °2θ; or (c) peaks at 2θ values essentially the same as shown in FIG. 1;

In one embodiment, Form 1 has a PXRD pattern comprising one or more peaks at 2θ values selected from the group consisting of: 4.8, 10.6, 14.3, 19.1 and 19.7 °2θ±0.2 °2θ. In another embodiment, Form 1 has a PXRD pattern comprising two or more peaks at 2θ values selected from the group consisting of: 4.8, 10.6, 14.3, 19.1 and 19.7 °2θ±0.2 °2θ. In another embodiment, Form 1 has a PXRD pattern comprising three or more peaks at 2θ values selected from the group consisting of: 4.8, 10.6, 14.3, 19.1 and 19.7 °2θ±0.2 °2θ. In another embodiment, Form 1 has a PXRD pattern comprising four or more peaks at 20 values selected from the group consisting of: 4.8, 10.6, 14.3, 19.1 and 19.7 °2θ±0.2 °2θ.

In one embodiment, Form 1 has a PXRD pattern comprising peaks at 2θ values of: 4.8, 14.3 and 19.7 °2θ±0.2 °2θ. In one embodiment, Form 1 has a PXRD pattern comprising peaks at 2θ values of: 4.8, 10.6, 14.3 and 19.7 °2θ±0.2 °2θ. In one embodiment, Form 1 has a PXRD pattern comprising peaks at 2θ values of: 4.8, 10.6, 14.3, 19.1 and 19.7 °2θ±0.2 °2θ.

In one embodiment, Form 1 has a PXRD pattern further comprising a peak at the 2θ value of: 4.8 °2θ±0.2 °2θ. In one embodiment, Form 1 has a PXRD pattern further comprising a peak at the 2θ value of: 10.6 °2θ±0.2 °2θ. In one embodiment, Form 1 has a PXRD pattern further comprising a peak at the 2θ value of: 14.3 °2θ±0.2 °2θ. In one embodiment, Form 1 has a PXRD pattern further comprising a peak at the 2θ value of: 19.1 °2θ±0.2 °2θ. In one embodiment, Form 1 has a PXRD pattern further comprising a peak at the 2θ value of: 19.7 °2θ±0.2 °2θ.

In some embodiments, the disclosure provides a pharmaceutical composition comprising the crystalline free base form of 4-((6-(2,2-difluoroethyl)-8-((1R,2R)-2-hydroxy-2-methylcyclopentyl)-7-oxo-7,8-dihydropyrido[2,3-d]pyrimidin-2- yl)amino)piperidine-1-sulfonamide (Form 1) according to any of the embodiments described herein, and a pharmaceutically acceptable carrier or excipient.

In another aspect, the disclosure provides method of treating abnormal cell growth in a mammal, preferably a human, comprising administering to the mammal a therapeutically effective amount of Form 1 according to any of the embodiments described herein.

In another aspect, the disclosure provides method of treating abnormal cell growth in a mammal, preferably a human, comprising administering to the mammal a therapeutically effective amount of a pharmaceutical composition comprising the Form 1 according to any of the embodiments described herein.

In another aspect, the disclosure provides uses of Form 1 according to any of the embodiments described herein in treating abnormal cell growth in a mammal, preferably a human.

In another aspect, the disclosure provides uses of Form 1 according to any of the embodiments described herein in the manufacture of a medicament for use in a treating abnormal cell growth in a mammal, preferably a human.

In frequent embodiments of the methods, compositions and uses described herein, the abnormal cell growth is cancer.

### Stereoisomers

The compound of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, the compound of the disclosure containing one or more asymmetric carbon atoms may exist as two or more stereoisomers.

The pharmaceutically acceptable salts of the compound of the disclosure may also contain a counterion which is optically active or racemic.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compound of the disclosure (and chiral precursors thereof) may be obtained in enantiomerically-enriched form using chromatography, typically HPLC Concentration of the eluate affords the enriched mixture. Chiral chromatography using sub-and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art; see, for example, Stereochemistry of Organic Compounds by E. L. Eliel and S. H. Wilen (Wiley, 1994).

### Tautomerism

Where structural isomers are interconvertible via a low energy barrier, tautomeric isomerism ('tautomerism') may occur. This may take the form of proton tautomerism in the compound of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compound of the disclosure has been drawn herein in a single tautomeric form, all possible tautomeric forms are included within the scope of the disclosure.

### Isotopes

The present disclosure includes all pharmaceutically acceptable isotopically-labeled compound of the disclosure wherein one or more atoms are replaced by atoms having the same atomic number, but an atomic mass or mass number different from the atomic mass or mass number which predominates in nature.

Examples of isotopes suitable for inclusion in the compound of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compound of the disclosure, for example those incorporating a radioactive isotope, are useful in one or both of drug or substrate tissue distribution studies. The radioactive isotopes, such as, tritium and ¹⁴C, are particularly useful for this purpose in view of their ease of incorporation and ready means of detection. Substitution with positron emitting isotopes, such as ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, metabolic sites on the compounds of the disclosure are deuterated.

Isotopically-labeled compound of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

### Prodrugs

The compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs.’ Further information on the use of prodrugs may be found in The Expanding Role of Prodrugs in Contemporary Drug Design and Development, Nature Reviews Drug Discovery, 17, 559-587 (2018) (J. Rautio et al.).

Prodrugs in accordance with the disclosure may, for example, be produced by replacing appropriate functionalities present in compound of the disclosure with certain moieties known to those skilled in the art as ‘pro-moieties’ as described, for example, in ‘Design of Prodrugs’ by H. Bundgaard (Elsevier, 1985).

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in the compound of the disclosure; (d) a thioester, thiocarbonate, thiocarbamate or sulfide derivatives of a thiol group when present in the compound of the disclosure; or (e) an oxime or imine derivative of a carbonyl group when present in the compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., tBuC(═O)OCH₂—);
(ii) when a compound of the disclosure contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the disclosure contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the disclosure contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

### Metabolites

Also included within the scope of the disclosure are active metabolites of the compound of the disclosure, that is, compounds formed in vivo upon administration of the drug, often by oxidation or dealkylation. Some examples of metabolites in accordance with the disclosure include, but are not limited to:
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (—Ph→—PhOH);
(vi) where the compound of the disclosure contains an amide group, a carboxylic acid derivative thereof (—CONH₂→COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the disclosure comprises pharmaceutical compositions.

A “pharmaceutical composition” refers to a mixture of the compound of the disclosure, or a pharmaceutically acceptable salt, solvate, hydrate or prodrug thereof as an active ingredient, and at least one pharmaceutically acceptable excipient.

The term “excipient” is used herein to describe any ingredient other than the compound(s) of the disclosure. The choice of excipient will to a large extent depend on factors such as the mode of administration, the effect of the excipient on solubility and stability, and the nature of the dosage form. “Excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The composition of the disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. In another embodiment, the oral administration may be in a powder or granule form. In another embodiment, the oral dosage form is sub-lingual, such as, for example, a lozenge. In such solid dosage forms, the compound of the disclosure is ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compound of the disclosure is conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 1.0, 5, 10, 15, 25, 50, 75, 100, 125, 150, 175, 200, 250, 500 or 1000 milligrams of the active ingredient for the symptomatic adjustment of the dosage to the patient. A medicament typically contains from about 1 mg to about 1000 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously, doses may range from about 0.01 to about 10 mg/kg/minute during a constant rate infusion.

Liposome containing the compound of the disclosure may be prepared by methods known in the art (See, for example, Chang, H.I.; Yeh, M.K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The compound of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate), or 'poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. The compound of the disclosure is generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. The compound of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah, et al.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the disclosure include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, the compound of the disclosure is administered in an amount effective to treat a condition as described herein. The compound of the disclosure may be administered as compound per se, or alternatively, as a pharmaceutically acceptable salt.

The compound of the disclosure is administered by any suitable route in the form of a pharmaceutical composition adapted to such a route, and in a dose effective for the treatment intended. The compound of the disclosure may be administered orally, rectally, vaginally, parenterally, topically, intranasally, or by inhalation.

The compound of the disclosure may be administered orally. Oral administration may involve swallowing, so that the compound enters the gastrointestinal tract, or buccal or sublingual administration may be employed by which the compound enters the bloodstream directly from the mouth.

In another embodiment, the compound of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compound of the disclosure may also be administered topically to the skin or mucosa, that is, dermally or transdermally. In another embodiment, the compound of the disclosure may also be administered intranasally or by inhalation. In another embodiment, the compound of the disclosure may be administered rectally or vaginally. In another embodiment, the compound of the disclosure may also be administered directly to the eye or ear.

The dosage regimen for the compound of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. In another embodiment, total daily dose of the compound of the disclosure is from about 0.1 to about 50 mg/kg, and in another embodiment, from about 0.5 to about 30 mg/kg. It is not uncommon that the administration of the compound of the disclosure will be repeated a plurality of times in a day (typically no greater than 4 times). Multiple doses per day typically may be used to increase the total daily dose, if desired.

### Therapeutic Methods and Uses

The compound of the disclosure may inhibit the activities of CDKs, including CDK2, CDK4 and/or CDK6, thereby effecting biological functions. Accordingly, the compound of the disclosure may be useful in the treatment, prevention, suppression, and amelioration of diseases such as cancers, disorders and conditions mediated by any of CDK2, CDK4 and/or CDK6, or a combination thereof.

In one aspect, the disclosure provides a method for the treatment of abnormal cell growth in a subject comprising administering to the subject a therapeutically effective amount of a compound of the disclosure, or a pharmaceutically acceptable salt thereof. In frequent embodiments, the abnormal cell growth is cancer.

In another aspect, the disclosure provides a method of inhibiting cancer cell proliferation in a subject, comprising administering to the subject a compound of the disclosure, or a pharmaceutically acceptable salt thereof, in an amount effective to inhibit cell proliferation.

In another aspect, the disclosure provides a method of inhibiting cancer cell invasiveness in a subject, comprising administering to the subject a compound of the disclosure, or a pharmaceutically acceptable salt thereof, in an amount effective to inhibit cell invasiveness.

In another aspect, the disclosure provides a method of inducing apoptosis in cancer cells in a subject, comprising administering to the subject a compound of the disclosure, or a pharmaceutically acceptable salt thereof, in an amount effective to induce apoptosis.

In some embodiments of the methods provided herein, the abnormal cell growth is cancer, wherein the cancer is selected from the group consisting of breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer and thyroid cancer. In further embodiments of the methods provided herein, the cancer is selected from the group consisting of breast cancer, ovarian cancer, bladder cancer, uterine cancer, prostate cancer, lung cancer, esophageal cancer, liver cancer, pancreatic cancer and stomach cancer. In some such embodiments, the cancer is characterized by amplification or overexpression of CCNE1 and/or CCNE2.

In some embodiments, the cancer is selected from the group consisting of breast cancer and ovarian cancer. In some such embodiments, the cancer is breast cancer or ovarian cancer characterized by amplification or overexpression of CCNE1 and/or CCNE2. In some such embodiments, the cancer is (a) breast cancer or ovarian cancer; (b) characterized by amplification or overexpression of cyclin E1 (CCNE1) or cyclin E2 (CCNE2); or (c) both (a) and (b).

In some embodiments, the cancer is ovarian cancer. In some such embodiments, the ovarian cancer is characterized by amplification or overexpression of CCNE1 and/or CCNE2. In some embodiments, the ovarian cancer is advanced or metastatic breast cancer.

In other embodiments, the cancer is breast cancer.

In some embodiments, the breast cancer is estrogen receptor (ER)-positive (ER+)/hormone receptor (HR)-positive (HR+).

In some embodiments, the breast cancer is human epidermal growth factor receptor 2 (HER2)-negative (HER2-).

In some embodiments, the breast cancer is ER-positive/HR-positive.

In some embodiments, the breast cancer is HER2-positive.

In some embodiments, the breast cancer is triple negative breast cancer (TNBC).

In some embodiments, the breast cancer is inflammatory breast cancer.

In some embodiments, the breast cancer is endocrine resistant breast cancer, trastuzumab resistant breast cancer, or breast cancer demonstrating primary or acquired resistance to CDK4/CDK6 inhibition.

In some embodiments, the breast cancer is advanced or metastatic breast cancer. In some embodiments of each of the foregoing, the breast cancer is characterized by amplification or overexpression of CCNE1 and/or CCNE2.

In some embodiments, the compound of the disclosure is administered as first line therapy. In other embodiments, the compound of the disclosure is administered as second (or later) line therapy. In some embodiments, the compound of the disclosure is administered as second (or later) line therapy following treatment with an endocrine therapeutic agent and/or a CDK4/CDK6 inhibitor. In some embodiments, the compound of the disclosure is administered as second (or later) line therapy following treatment with an endocrine therapeutic agent. In some embodiments, the compound of the disclosure is administered as second (or later) line therapy following treatment with a CDK4/CDK6 inhibitor. In some embodiments, the compound of the disclosure is administered as second (or later) line therapy following treatment with one or more chemotherapy regimens, e.g., including taxanes or platinum agents. In some embodiments, the compound of the disclosure is administered as second (or later) line therapy following treatment with HER2 targeted agents, e.g., trastuzumab. In some embodiments, the compound of the disclosure is administered after failure of the treatment with an endocrine therapeutic agent.

### Co-Administration

The compound of the disclosure may be used alone, or in combination with one or more other therapeutic agents. The disclosure provides any of the uses, methods or compositions as defined herein wherein the compounds of Formula (I), or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

The compounds of Formula (I) and the one or more other therapeutic agents may be administered as a fixed or non-fixed combination of the active ingredients. The term “fixed combination” means the compounds of Formula (I), or a pharmaceutically acceptable salt thereof, and the one or more therapeutic agents, are both administered to a subject simultaneously in a single composition or dosage. The term “non-fixed combination” means that The compounds of Formula (I), or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

Classes of additional chemotherapeutic agents, which can be administered in combination with a compound of this disclosure, include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists; IL-2 receptor agonist (recombinant cytokines or agonists for cytokine receptors); and anti-sense oligonucleotides or oligonucleotides derivatives that inhibit expression of genes implicated in abnormal cell proliferation or tumor growth.

Other additional chemotherapy agents include not only taxanes or platinum agents but also HER2 targeted agents, e.g., trastuzumab.

In another embodiment, such additional anti-cancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, spindle poison plant alkaloids, MCT4 inhibitors; MAT2a inhibitors; alk/c-Met/ROS inhibitors (including crizotinib or lorlatinib); mTOR inhibitors (including temsirolimus or gedatolisib); src/abl inhibitors (including bosutinib); cyclin-dependent kinase (CDK) inhibitors (including palbociclib); erb inhibitors (including dacomitinib); PARP inhibitors (including talazoparib); SMO inhibitors (including glasdegib); EGFR T790M inhibitors; PRMT5 inhibitors; TGFβR1 inhibitors; growth factor inhibitors; cell cycle inhibitors, biological response modifiers; enzyme inhibitors; and cytotoxics.

In another embodiment, such additional anti-cancer therapeutic agents include compounds derived from an anti-angiogenesis agent, including for example tyrosine kinase/vascular endothelial growth factor (VEGF) receptor (VEGFR) inhibitors (including sunitinib, axitinib, sorafenib, and tivozanib), TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™), axitinib (Inlyta™), SU 14813 (Pfizer), and AG 13958 (Pfizer). Additional anti-angiogenesis agents include vatalanib (CGP 79787), pegaptanib octasodium (Macugen™), vandetanib (Zactima™), PF-0337210 (Pfizer), SU 14843 (Pfizer), AZD 2171 (AstraZeneca), ranibizumab (Lucentis™) Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™) deracoxib (SC 59046), lumiracoxib (Preige™), valdecoxib (Bextra™), rofecoxib (Vioxx™) iguratimod (Careram™), IP 751 (Invedus), SC-58125 (Pharmacia) and etoricoxib (Arcoxia™) Yet further anti-angiogenesis agents include exisulind (Aptosyn™), salsalate (Amigesic™) diflunisal (Dolobid™), ibuprofen (Motrin™), ketoprofen (Orudis™), nabumetone (Relafen™) piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon). Yet further anti-angiogenesis agents include acitretin (Neotigason™), plitidepsin (aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment, such additional anti-cancer therapeutic agents include compounds derived from hormonal agents and antagonists. Examples include where anti-hormonal agents act to regulate or inhibit hormone action on tumors such as anti-estrogens and selective estrogen receptor modulators (SERMs), and a selective estrogen receptor degrader (SERD) including tamoxifen, raloxifene, droloxifene, 4-hydroxytamoxifen, trioxifene, keoxifene, LY117018, onapristone, toremifene (Fareston), and fulvestrant. Examples also include aromatase inhibitors that inhibit the enzyme aromatase, which regulates estrogen production in the adrenal glands, and include compounds like 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin.

In another embodiment, such additional anti-cancer therapeutic agents include compounds derived from signal transduction inhibitors, such as inhibitors of protein tyrosine kinases and/or serine/threonine kinases: a signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK (including binimetinib (Mektovi™)), c-Kit inhibitors, FLT-3 inhibitors, K-Ras inhibitors, PI3 kinase inhibitors, JAK inhibitors, STAT inhibitors, Raf kinase inhibitors, BRAF (including encorafenib (Braftovi™)), Akt inhibitors, mTOR inhibitor, P70S6 kinase inhibitors, inhibitors of the WNT pathway and multi-targeted kinase inhibitors.

In another embodiment, such additional anti-cancer therapeutic agents include docetaxel, paclitaxel, paclitaxel protein-bound particles, cisplatin, carboplatin, oxaliplatin, capecitabine, gemcitabine or vinorelbine.

In another embodiment, such additional anti-cancer therapeutic agents include compounds derived from an epigenetic modulator, where examples include an inhibitor of EZH2 (including PF-06821497), SMARCA4, PBRM1, ARID1A, ARID2, ARID1B, DNMT3A, TET2, MLL1/2/3, NSD1/2, SETD2, BRD4, DOTI L, HKMTsanti, PRMT1-9, LSD1, UTX, IDH1/2 or BCL6.

In another embodiment, such additional anti-cancer therapeutic agents include compounds that are immuno-oncology agents, including immunomodulatory agents.

In another embodiment, combinations with pattern recognition receptors (PRRs) are contemplated. PRRs are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein.

The STING protein functions as both a cytosolic DNA sensor and an adaptor protein in Type 1 interferon signaling. The terms “STING” and “stimulator of interferon genes” refer to any form of the STING protein, as well as variants, isoforms, and species homologs that retain at least a part of the activity of STING. Unless indicated differently, such as by specific reference to human STING, STING includes all mammalian species of native sequence STING, e.g., human, monkey, and mouse STING is also known as—TMEM173.

“STING agonist” as used herein means, any molecule, which upon binding to STING, (1) stimulates or activates STING, (2) enhances, increases, promotes, induces, or prolongs an activity, function, or presence of STING, or (3) enhances, increases, promotes, or induces the expression of STING. STING agonists useful in the any of the treatment method, medicaments and uses of the present disclosure include, for example, nucleic acid ligands which bind STING.

Examples of STING agonists that are useful in the treatment methods, medicaments, and uses of the present disclosure include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as WO2019027858, WO20180093964, WO2017175156, WO2017175147.

Therapeutic antibodies may have specificity against a variety of different antigens. For example, therapeutic antibodies may be directed to a tumor associated-antigen, such that binding of the antibody to the antigen promotes death of the cell expressing the antigen. In other example, therapeutic antibodies may be directed to an antigen on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

In another embodiment, such additional anti-cancer therapeutic agents include antibodies that would be blocking or inhibitory at the target: CTLA-4 (including ipilimumab or tremelimumab), PD-1 or PD-L1 (including atezolizumab, avelumab, cemiplimab, durvalumab, nivolumab, sasanlimab, or pembrolizumab), LAG-3, TIM-3, or TIGIT.

In another embodiment, such additional anti-cancer therapeutic agents include antibodies that are agonists of 4-1BB, OX40, GITR, ICOS, or CD40.

In another embodiment the anti-cancer therapy may be a CAR-T-cell therapy. Examples of a therapeutic antibody include: an anti-OX40 antibody, an anti-4-1BB antibody, an anti-HER2 antibody (including an anti-HER2 antibody-drug conjugate (ADC)), a bispecific anti-CD47/anti-PD-L1 antibody, and a bispecific anti-P-cadherin/anti-CD3 antibody. Examples of cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof. Exemplary immunomodulating agents that may be incorporated in an ADC include gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-15, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta. and -.gamma), the stem cell growth factor designated “S 1 factor,” erythropoietin and thrombopoietin, or a combination thereof.

Additional examples of therapeutic antibodies may include the following antigens where exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. see U.S. Pat. No. 9,969,809]; BTN1A1 (e.g. see WO2018222689); CA-125 (e.g. abagovomab); Carboanhydrase IX; CCR2; CCR4 (e.g. mogamulizumab); CCRS (e.g. leronlimab); CCR8; CD3 [e.g. blinatumomab (CD3/CD19 bispecific), CD3/P-cadherin bispecific, CD3/BCMA bispecific] CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD123; CD276/B7-H3 (e.g. omburtamab); CDH17; CEA; ClhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); GD2 (e.g. dinutuximab, 3F8); GD3; GITR; GloboH; GM1; GM2; HER2/neu [e.g. margetuximab, pertuzumab, trastuzumab; ado-trastuzumab emtansine, trastuzumab duocarmazine, [see U.S. Pat. No. 8,828,401]; HER3; HER4; ICOS; IL-10; ITG-AvB6; LAG-3 (e.g. relatlimab); Lewis-Y; LG; Ly-6; M-CSF [see U.S. Pat. No. 7,326,414]; MCSP; mesothelin; MUC1; MUC2; MUC3; MUC4; MUC5AC; MUC5B; MUC7; MUC16; Notch1; Notch3; Nectin-4 (e.g. enfortumab vedotin); OX40 [see U.S. Pat. No. 7,960,515]; P-Cadherein [see WO2016/001810]; PCDHB2; PDGFRA (e.g. olaratumab); Plasma Cell Antigen; PolySA; PSCA; PSMA; PTK7 [see U.S. Pat. No. 9,409,995]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7,10-tetraazacyclododecane-N,N′,N″,N″′-tetraacetic acid (DOTA), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

The disclosure also concerns the use of radiation in combination with any anti-cancer therapeutic agent administered herein. More specifically, compound of the disclosure can be administered in combination with additional therapies, such as radiation therapy and/or chemotherapy.

These agents and compound of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the disclosure provides kits comprising the compounds of Formula (I) or pharmaceutical compositions comprising the compounds of Formula (I). A kit may include, in addition to the compounds of Formula (I) or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents.

In yet another embodiment, the disclosure comprises kits that are suitable for use in performing the methods of treatment described herein. In one embodiment, the kit contains a first dosage form comprising one or more of the compound of the disclosure in quantities sufficient to carry out the methods of the disclosure. In another embodiment, the kit comprises one or more compound of the disclosure in quantities sufficient to carry out the methods of the disclosure and a container for the dosage and a container for the dosage.

### Synthetic Methods

The compounds of Formula (I) may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compound of the disclosure. It will be further appreciated that it may be necessary or desirable to carry out the transformations in a different order from that described in the schemes, or to modify one or more of the transformations, to provide the desired compounds of Formula (I).

In the preparation of compound of the disclosure it is noted that some of the preparation methods useful for the preparation of the compounds described herein may require protection of remote functionality (e.g., a primary amine, secondary amine, carboxyl, etc. in a precursor of Formula (I)). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in the compounds of Formula (I).

### General Experimental Details

¹H Nuclear Magnetic Resonance (NMR) spectra was recorded on Bruker XWIN-NMR (400 or 700 MHz) spectrometer. ¹H resonance is reported in parts per million (ppm) downfield from tetramethylsilane. ¹H NMR data are reported as multiplicity (e.g., s, singlet; d, doublet; t, triplet; q, quartet; quint, quintuplet; dd, doublet of doublets; dt, doublet of triplets; br s, broad singlet). For spectra obtained in CDCl₃, DMSO-d₆, and CD₃OD, the residual protons (7.27, 2.50, and 3.31 ppm, respectively) were used as the internal reference. All observed coupling constants, J, are reported in Hertz (Hz). Exchangeable protons are not always observed.

Optical rotations were determined on a Jasco P-2000 or a Rudolph Autopol IV polarimeter. All final compounds were purified to 95% purity, unless otherwise specified.

Mass spectra, MS (m/z), were recorded using either electrospray ionization (ESI) or atmospheric pressure chemical ionization (APCI). Where relevant and unless otherwise stated, the m/z data provided are for isotopes ¹⁹F, ³⁵Cl, ⁷⁹Br and ¹²⁷I.

The nomenclature is written as described by IUPAC (International Union of Pure and Applied Chemistry generated within Perkin Elmers Chemdraw 18.0.0.231. The naming convention provided with Perkin Elmers Chemdraw 18.0.0.231 is well known by those skilled in the art and it is believed that the naming convention provided with Perkin Elmers Chemdraw 18.0.0.231 generally comports with the IUPAC (International Union for Pure and Applied Chemistry) recommendations on Nomenclature of Organic Chemistry and the CAS Index rules.

The following abbreviations are used throughout the Examples: “Ac” means acetyl, “OAc” means acetoxy, “aq” means aqueous, “DCM” (CH₂Cl₂) means methylene chloride, “d.i.” means deionized, “DIEA” means diisopropyl ethyl amine, “DMSO” means dimethylsulfoxide, “EtOAc” means ethyl acetate, “EtOH” means ethanol, “HOAc” or “AcOH” means acetic acid, “i-Pr” or “Pr” means isopropyl, “LiHMDS” means lithium hexamethyldisilazide (lithium bis(trimethylsilyl)amide), “Me” means methyl, “MeOH” means methanol, “MS” means mass spectrometry, “MTBE” means methyl tert-butyl ether, “THF” means tetrahydrofuran, “2-MeTHF” means 2-methyltetrahydrofuran, “PXRD” means powder X-ray Diffraction, “oxone” is the potassium salt of peroxymonosulfuric acid, “SFC” means supercritical fluid chromatography, “TLC” means thin layer chromatography, “r.b.” means round bottom, “Rf” means retention fraction, “˜” means approximately, “rt” means room temperature, “h” means hours, “min” means minutes, “equiv” means equivalents, “sat.” means saturated.

Compounds 1-4 are potent inhibitors of CDK2, CDK4 and CDK6 having the formula:

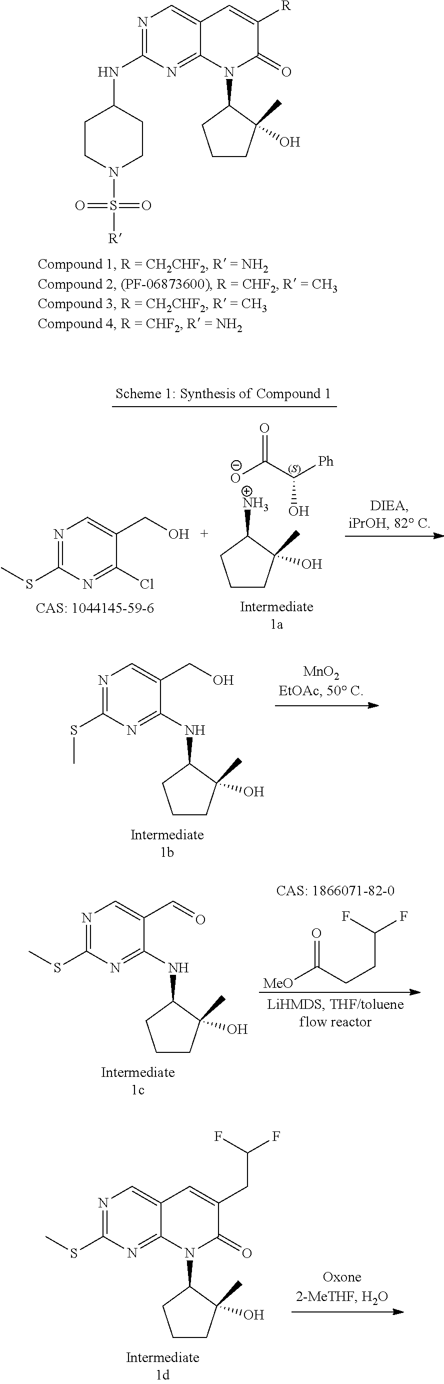

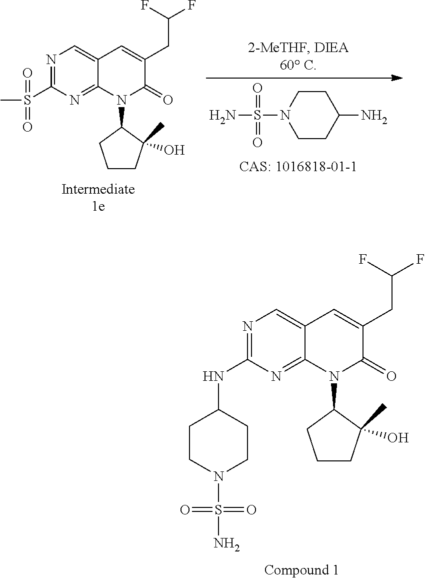 [SMILES: C[C@@]1(O)CCC[C@H]1n1c(=O)c(CC(F)F)cc2cnc(S(C)(=O)=O)nc21 ; NC1CCN(S(N)(=O)=O)CC1 ; C[C@@]1(O)CCC[C@H]1n1c(=O)c(CC(F)F)cc2cnc(NC3CCN(S(N)(=O)=O)CC3)nc21]

### Compound 1: 4-({6-(2,2-difluoroethyl)-8-[(1R,2R)-2-hydroxy-2-methylcyclopentyl]-7-oxo-7,8-dihydropyrido[2,3-d]pyrimidin-2-yl}amino)piperidine-1-sulfonamide

A 2 L reaction flask equipped with an overhead stirrer was charged with Intermediate 1a (112 g, 420 mmol), and iPrOH (240 mL). Intermediate 1a was prepared according to the procedure described in Duan, S. et al., Organic Process Research & Development 2020, 24 (11), 2734-2744. The reaction was stirred at rt for about 10 min., and DIEA (244 g, 1890 mmol) was added dropwise. After stirring, CAS 1044145-59-6 (80 g, 420 mmol) was added and the reaction head space was purged with nitrogen. The reaction temperature was increased to 82° C. and held at that temperature for 27 h while stirring. After cooling to rt, the reaction was concentrated to ˜150 mL total volume. Water (200 mL) was added and the aq. layer was extracted with 2-MeTHF (500 mL×3). The combined organic extract was washed with 30% aq. K ₂CO₃ (280 mL×2). The organic layer was concentrated to —150 mL total volume and MTBE (500 mL) was added. The resulting slurry was stirred at 15˜25° C. for 14 h and then cooled to 0° C. The resulting solid was filtered. After the wet cake was dried in a vacuum oven at 45° C., (1R,2R)-2-{[5-(hydroxymethyl)-2-(methylsulfanyl)pyrimidin-4-yl]amino}-1-methylcyclopentanol Intermediate 1b (86 g, 76%) was obtained as an off white solid. ¹H NMR (400 MHz, CDCl₃) δ=7.76; (s, 1H), 6.01 (d, J =4.6 Hz, 1H), 5.31; (br s, 1H), 4.55; (s, 2H), 4.26; (ddd, J=5.7, 8.2, 10.5 Hz, 1H), 2.50; (s, 3H), 2.21; (ddd, J=3.5, 8.2, 12.1 Hz, 1H), 1.97; (dt, J=3.5, 7.7 Hz, 1H), 1.89-1.76; (m, 2H), 1.75-1.63; (m, 1H), 1.60-1.50; (m, 2H), 1.11; (s, 3H). MS: 270 [M+H]⁺. Optical rotation: [α]_{D}²²+37.7 (c 1.0, MeOH).

### MnO₂ Oxidation to Afford Intermediate 1c

Intermediate 1b (133 g, 495 mmol) was dissolved in THF (670 mL). Activated MnO 2 (151 g, 1740 mmol) was added and the reaction was stirred at 55° C. for 26 h. The MnO₂ by-products were filtered off using Celite and the Celite layer was rinsed with THF (1340 mL). The filtrate was concentrated to ˜150 mL and heptane (670 mL) was added. The heptane was concentrated to ˜150 mL and the heptane treatment was repeated two more times each time concentrating to ˜150 mL. The solid obtained was filtered, washed with heptane (˜200 mL) and the wet cake was dried in a vacuum oven at 45° C. for 20 h to afford 4-{[(1R,2R)-2-hydroxy-2-methylcyclopentyl]amino}-2-(methylsulfanyl)pyrimidine-5-carbaldehyde, Intermediate 1c (132 g, 95%) as a solid. ¹H NMR (400 MHz, CDCl₃) δ=9.73; (s, 1H), 8.66; (br s, 1H), 8.35; (s, 1H), 4.39; (ddd, J=6.5, 8.2, 9.6 Hz, 1H), 4.16; (s, 1H), 2.57; (s, 3H), 2.33-2.22; (m, 1H), 2.03-1.92; (m, 1H), 1.89-1.68; (m, 3H), 1.68-1.56; (m, 1H), 1.17; (s, 3H), MS: 268 [M+H]+. Optical rotation [α]_{D}²²+12.7 (c 1.0, CHCl₃).

### Aldol Reaction Using a Flow Reactor to Afford Intermediate 1d

Reactor #1 was charged with Intermediate 1c (80.0 g, 299 mmol), CAS 1866071-82-0 (70.3 g, 509 mmol), THF (1.44 L) and toluene (320 mL). Reactor #2 was charged with LiHMDS (1.05 L of 1 M in THF, 1.05 mol) and toluene (184 mL). Reactor #3 was charged with water (100 mL). Peristaltic pumps fed the solutions from reactors #1 and #2 into pre-cooled coils maintained at temperatures between 15-25° C. The flow rate from reactor #1 was set at 2.82 mL per min, the flow rate from reactor #2 was set at 1.84 mL per min and flow rate from reactor #3 was set at 0.6 mL per min. After the flow from reactors #1 and #2 were mixed at temperatures between 15-25° C. the mixture flowed into a stirred tank where the flow from reactor #3 was also being added. The stirred tank is where quenching of the reaction occurred. The temperature of the stirred tank was maintained at between 0-10° C. The quenched mixture was stirred for 0.5-3 h at between 0-10° C. From two flow batches of the reaction described above at comparable scale, 0.718 mol of the quenched reaction mixture was obtained which was worked up in the following manner. First, the lower aq. layer was drained from the mixture. Then, 2 N HCl (80 mL) was added and the mixture was stirred for 20 min. After separating the layers, the lower layer was drained off. The resulting organic layer was concentrated to ˜720 mL. A solvent swap to MTBE was performed by adding 400 mL MTBE and concentrating to 720 mL twice. After the solvent swap, 400 mL of MTBE was added followed by 1 N HCl (160 mL). The mixture was stirred for 30 min and the layers separated. The lower layer was drained off and 1 N HCl (160 mL) was added again. The mixture was stirred for 30 min and the layers separated. The lower layer was drained off. 7% aq. NaHCO₃ (160 mL) was added, the mixture was stirred for 30 min and the layers separated. The organic layer was concentrated to ˜720 mL and 2-MeTHF (400 mL) was added. The resulting mixture was concentrated to ˜720 mL and 2-MeTHF (400 mL) was added again. After concentrating to ˜720 mL, 2-MeTHF (400 mL) was added. The solid that formed was allowed to sit for 2-5 h and was collected to afford 6-(2,2-difluoroethyl)-8-[(1R,2R)-2-hydroxy-2-methylcyclopentyl]-2-(methylsulfanyl)pyrido[2,3- cipyrimidin-7(8H)-one, Intermediate 1d (240 g, 94%) as a solid. ¹H NMR (400 MHz, CHLOROFORM-d) d 8.63; (s, 1H), 7.59; (s, 1H), 6.31-5.97; (m, 1H), 5.88; (t, J=8.6 Hz, 1H), 3.15; (dt, J=4.5, 16.3 Hz, 2H), 2.87-2.72; (m, 1H), 2.64; (s, 3H), 2.32-2.21; (m, 2H), 2.12-2.06; (m, 1H), 2.04-2.01; (m, 1H), 2.00-1.91; (m, 1H), 1.91-1.82; (m, 1H), 1.14; (s, 3H), MS: 356.1 [M+H]⁺.

### Oxone Oxidation to Afford Intermediate 1d

Intermediate 1d (300 g, 0.844 mol) was dissolved in 2-MeTHF (2.7 L) and water (1.05 L) was added. The temperature was cooled to between 0-10° C. Oxone (1.3 kg, 2.1 mol) was added and the reaction was warmed to between 20-30° C. Stirring at this temperature was continued for 16 h. Water (1.5 L) was added and stirring was continued for 15 min. After separating the layers, the lower aqueous layer was drained. Then, 5% aq. NaHSO₃ (900 mL) was added and stirring was continued for 15 min. The lower aqueous layer was drained and 5% aq. Na₂SO₄ (900 mL) was added stirring for 15 min. The lower aqueous layer was drained. 30 g of activated carbon was added to the organic phase and stirring was continued for 16 h. The organic layer was filtered through Celite, washing the filter cake with 2-MeTHF (600 mL). The organic layer was concentrated and MTBE (1.2 L) was added. The organic layer was concentrated and MTBE (1.2 L) was added again. The organic layer was concentrated and MTBE (1.2 L) was added again. The mixture was heated to between 30-40° C. and stirred for 1 h. While stirring, the reaction was cooled to between 10-20° C. and stirred at that temperature for 2 h. The solid that formed was collected washing with MTBE (600 mL) and dried in a vacuum oven at 40° C. for 16 h. The resulting solid was recrystallized by dissolving it in 2-MeTHF (600 mL), concentrating and adding MTBE (1.2 L). The MTBE was removed in vacuo at 40° C. MTBE (1.2 L) was added and the organic layer was concentrated. MTBE (1.2 L) was added again the mixture was heated to between 30-40° C. and stirred for 1 h. While stirring, the reaction was cooled to between 10-20° C. and stirred at that temperature for 2 h. The solid that formed was collected washing with MTBE (600 mL) and dried in a vacuum oven at 40° C. for 16 h to afford 6-(2,2-difluoroethyl)-8-[(1R,2R)-2-hydroxy-2-methylcyclopentyl]-2-(methanesulfonyhpyrido[2,3-d]pyrimidin-7(81-0-one, Intermediate 1e (211 g, 64%) as an off-white solid.

### SnAr reaction to afford Compound 1

To a stirred solution of compound Intermediate 1e (17 g, 44 mmol) in 2-MeTHF (350 mL) was added CAS 1016818-01-1 (19.7 g, 110 mmol) at 20° C. The reaction was heated to 60° C. and stirred for 16 h. TLC (DCM:MeOH=10:1) showed the reaction was complete. The mixture was diluted by water (400 mL) and extracted with EtOAc (4×300 mL). The combined organic layers were washed with brine (100 mL), dried over MgSO₄, filtered and concentrated to give a crude product which was purified by flash chromatography using a gradient of 0-10% MeOH in DCM. This afforded 18 g of crude Compound 1 as a yellow solid which was further purified by prep-HPLC using a Xtimate C18 (150 mm*40 mm*5 mm) column and gradient elution of CH₃CN in water containing 0.2% formic acid. After the fractions were evaporated to remove most of the solvent, the remaining solution was dissolved in EtOAc (750 mL) and washed with satd. aq. NaHCO₃. The EtOAc layer was washed with brine (100 mL), dried over MgSO₄, filtered and concentrated to give (13.5 g, 78%) of 4-({6-(2,2-difluoroethyl)-8-[(1R,2R)-2-hydroxy-2-methylcyclopentyl]-7-oxo-7,8-dihydropyrido[2,3-d]pyrimidin-2-yl}amino)piperidine-1- sulfonamide, Compound 1 as a white EtOAc solvate.

### Conversion of the Solvate to the Anhydrous Solid Form of Compound 1

To obtain an anhydrous solid form, a 1 L three-neck r.b. flask was fitted with an internal thermometer, OH stirrer and heating source (oil bath). Solvated Compound 1 (10.9 g, 22.4 mmol) was added to the r.b. followed by 250 mL of d.i. water. Stirring was initiated while the oil bath was heated to give an internal temperature of 48° C. Stirring was continued for 4 days at around 600 rpm while the internal temperature was maintained at between 45-48° C. On day 4, a sample was taken out for PXRD which showed conversion to the desired anhydrous form. Because of the drying time prior to PXRD, the total stirring time for this batch was 5 days at 45-48° C. The entire batch was filtered on day 5, rinsing with water to transfer all the material into a Buchner funnel. Filtration proceeded slowly to afford a wet cake. The wet cake was compressed and suction filtration was continued for another 1 h. The material was placed on H-vac overnight. Anhydrous Compound 1 (9.6 g, 88%) was obtained. Elemental analysis and no observed residual solvent by ¹H NMR also supported identification of the anhydrous form. ¹H NMR (DMSO-d₆, 400 MHz, 80° C.) δ 8.57; (s, 1H), 7.69; (s, 1H), 7.40; (br d, 1H, J=6.8 Hz), 6.47; (s, 2H), 6.19; (tt, 1H, J=4.6, 57.2 Hz), 5.8-5.9; (m, 1H), 4.01; (s, 1H), 3.9-4.0; (m, 1H), 3.5-3.6; (m, 2H), 3.0-3.1; (m, 2H), 2.75; (tt, 2H, J=2.4, 11.7 Hz), 2.2-2.3; (m, 1H), 1.8-2.1; (m, 5H), 1.5-1.8; (m, 3H), 1.01; (s, 3H), MS: 487.2 [M+H]^{+, [α]}_{D}²²−20.3; (c 0.2, MeOH), Anal. Calcd for C₂₀H₂₈F₂N₆O₄S: C, 49.37; H, 5.80; N, 17.27; Found: C, 49.49; H, 5.65; N, 17.09; qNMR=98.0+/−1.8%, HPLC purity=98.9%.

The anhydrous Compound 1 prepared above was further characterized by powder X-ray diffraction (PXRD).

### Powder X-Ray Diffraction

Powder X-ray diffraction analysis was conducted using a Bruker AXS D8 Endeavor diffractometer equipped with a Cu radiation source. The divergence slit was set at 15 mm continuous illumination. Diffracted radiation was detected by a PSD-Lynx Eye detector, with the detector PSD opening set at 4.107 degrees. The X-ray tube voltage and amperage were set to 40 kV and 40 mA respectively. Data was collected at the Cu wavelength (CuK_{α̅}=1.5418λ) in the Theta-Theta goniometer from 3.0 to 40.0 degrees 2-Theta using a step size of 0.01 degrees and a step time of 1.0 second. The antiscatter screen was set to a fixed distance of 1.5 mm. Samples were rotated at 15/min during data collection. Samples were prepared by placing them in a silicon low background sample holder and rotated during collection. Data were collected using Bruker DIFFRAC Plus software. and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum.

Peaks with relative intensity of 3% were generally chosen. Typically, the peaks which were not resolved or were consistent with noise were not selected. A typical error associated with the peak position from PXRD stated in USP up to +/−0.2°2-Theta (USP-941).

The PXRD pattern of Compound 1 free base, Form 1, is shown in FIG. 1. A PXRD peak list and relative intensity data for Compound 1 free base, Form 1 (2-Theta °) is provided in Table 1 below.

TABLE 1
PXRD peak list for Form 1, anhydrous crystalline free base.

| Angle (2 theta) | Relative Intensity (%) |
|---|---|
| 4.8* | 100.0 |
| 10.6* | 38.0 |
| 14.3* | 84.5 |
| 17.0 | 11.4 |
| 17.2 | 5.2 |
| 17.4 | 4.5 |
| 18.4 | 15.4 |
| 19.1* | 41.9 |
| 19.7* | 66.8 |
| 20.2 | 10.8 |
| 21.8 | 3.1 |
| 22.9 | 3.4 |
| 23.9 | 10.8 |
| 25.8 | 3.9 |
| 28.8 | 4.4 |
| 34.4 | 3.6 |
| 35.2 | 4.3 |

Asterisked peak positions represent characteristic peaks of Form 1

### Synthesis of Compounds 2-4

Preparation of Compounds 2-4 were disclosed in International Patent Publication No. WO2018/033815 and in United States Patent Application No. 2018/0044344. Improved methods of preparation of Compound 2 (PF-06873600) have been disclosed in several publications. Freeman-Cook, et. al., J. Med. Chem., 2021, 64 (13), 9056-9077; Meng, D. et al., Cell Reports Physical Science, 2021, 2 (4), 100394. The contents of each of the foregoing documents are incorporated herein by reference in their entirety.

### Deuterated Analogs of Compound 1

The compounds shown in Table 2 are prophetic deuterated analogs (PDA) of Compound 1. The PDAs are predicted based on the metabolic profile of Compound 1 (obtained from both the metabolism assessment and MetaSite database).

TABLE 2
Deuterated Analogs of Compound 1
(II)

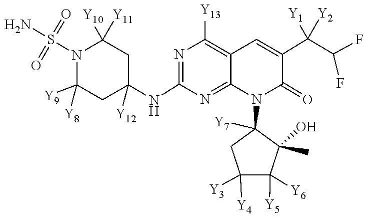

| PDA | Y₁ | Y₂ | Y₃ | Y₄ | Y₅ | Y₆ | Y₇ | Y₈ | Y₉ | Y₁₀ | Y₁₁ | Y₁₂ | Y₁₃ |
|---|---|---|---|---|---|---|---|---|---|---|---|---|---|
| 1 | D | D | H | H | H | H | H | H | H | H | H | H | H |
| 2 | H | H | D | D | H | H | H | H | H | H | H | H | H |
| 3 | H | H | H | H | D | D | H | H | H | H | H | H | H |
| 4 | H | H | H | H | H | H | D | H | H | H | H | H | H |
| 5 | H | H | H | H | H | H | H | D | D | H | H | H | H |
| 6 | H | H | H | H | H | H | H | H | H | D | D | H | H |
| 7 | H | H | H | H | H | H | H | H | H | H | H | D | H |
| 8 | H | H | H | H | H | H | H | H | H | H | H | H | D |

A preliminary assessment of the metabolism of Compound 1 was conducted using mouse, rat, rabbit, dog, monkey and human hepatocytes; human liver microsomes; plasma from monkey, dog and mouse studies following oral dosing. The primary metabolic pathway of Compound 1 was oxidation.

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.org); King, R., “Biotransformations in Drug Metabolism,” Ch.3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (lhasalimited.org/products/meteor-nexus.htm) offers prediction of metabolic pathways and metabolite structures using a range of machine learning models, which covers phase I and phase II biotransformations of small molecules.

PDA 1-6 in Table 2 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

A person with ordinary skill may make additional deuterated analogs of Compound 1 with different combinations of Y₁-Y₁₃ as provided in Table 2. Such additional deuterated analogs may provide similar therapeutic advantages that may be achieved by the deuterated analogs.

### Example 1: Biochemical Assays

Compound 1 and PF-06873600 were tested against highly purified, untagged CDK complexes using mobility shift assay (MSA).

### CDK2/Cyclin E1 Mobility Shift Assay

The purpose of the CDK2/Cyclin E1 assay is to evaluate the inhibition (% inhibition, Kᵢₐₚₚ and Kᵢ values) of Compound 1 and PF-06873600 by using a fluorescence-based microfluidic mobility shift assay. CDK2/Cyclin E1 catalyzes the production of ADP from ATP that accompanies the phosphoryl transfer to the substrate peptide FL-Peptide-18 (5-FAM-QSPKKG-CONH₂) (SEQ ID NO:1). (CPC Scientific, Sunnyvale, CA). The mobility shift assay electrophoretically separates the fluorescently labeled peptides (substrate and phosphorylated product) following the kinase reaction. Both substrate and product are measured, and the ratio of these values is used to generate % conversion of substrate to product by the LabChip EZ Reader. Wild-type full length CDK2/wild-type full length Cyclin E1 enzyme complex was produced in-house (baculoviral expression, LJIC-2080/LJIC-2103) and phosphorylated by CDK7/Cyclin H1/Mat1 enzyme complex with CDK2:CDK7 ratio of 50:1 (concentration mg/mL) in the presence of 10 mM MgCl₂ and 5 mM ATP at room temperature for one hour. Typical reaction solutions (50 μL final reaction volume) contained 2% DMSO (±inhibitor), 4 mM MgCl₂, 1 mM DTT, 150 μM ATP (ATP Kₘ=67.4 μM), 0.005% Tween-20, 3 μM FL-Peptide-18, and 0.36 nM (catalytically competent active site) phosphorylated wild-type full length CDK2/Cyclin E1 enzyme complex in 25 mM HEPES buffer at pH 7.15. The assay was initiated with the addition of ATP, following a fifteen minutes pre-incubation of enzyme and inhibitor at room temperature in the reaction mixture. The reaction was stopped after 45 minutes at room temperature by the addition of 50 μL of 80 mM EDTA, pH 7.5. The Kᵢ value was determined from the fit of the data to the Morrison tight-binding competitive inhibition equation with the enzyme concentration as a variable.

### CDK4/Cyclin D1 Mobility Shift Assay

The purpose CDK4/Cyclin D1 assay is to evaluate the inhibition (% inhibition, Kᵢₐₚₚ and Kᵢ values) in the presence of Compound 1 and PF-06873600 by using a fluorescence based microfluidic mobility shift assay. CDK4/Cyclin D1 catalyzes the production of ADP from ATP that accompanies the phosphoryl transfer to the substrate peptide 5-FAM-Dyrktide (5-FAM-RRRFRPASPLRGPPK) (SEQ ID NO:1). The mobility shift assay electrophoretically separates the fluorescently labeled peptides (substrate and phosphorylated product) following the kinase reaction. Both substrate and product are measured and the ratio of these values is used to generate % Conversion of substrate to product by the LabChip EZ Reader. Typical reaction solutions contained 2% DMSO (±inhibitor), 10 mM MgCl₂, 1 mM DTT, 3.5 mM ATP, 0.005% TW-20, 3 μM 5-FAM-Dyrktide, 3 nM (active sites) activated CDK4/Cyclin D1 in 40 mM HEPES buffer at pH 7.5.

Inhibitor Kᵢ determinations for activated CDK4/Cyclin D1 (2007 E1/2008+PO4) were initiated with the addition of ATP (50 μL final reaction volume), following an eighteen-minute preincubation of enzyme and inhibitor at 22° C. in the reaction mix. The reaction was stopped after 195 minutes by the addition of 50 μL of 30 mM EDTA. K, determinations were made from a plot of the fractional velocity as a function of inhibitor concentration fit to the Morrison equation with the enzyme concentration as a variable.

### CDK6/Cyclin D3 Mobility Shift Assay

The purpose of the CDK6/Cyclin D3 assay is to evaluate the inhibition (% inhibition, Kᵢₐₚₚ and Kᵢ values) in the presence of Compound 1 and PF-06873600 by using a fluorescence based microfluidic mobility shift assay. CDK6/Cyclin D3 catalyzes the production of ADP from ATP that accompanies the phosphoryl transfer to the substrate peptide 5-FAM-Dyrktide (5-FAM-RRRFRPASPLRGPPK) (SEQ ID NO:1). The mobility shift assay electrophoretically separates the fluorescently labeled peptides (substrate and phosphorylated product) following the kinase reaction. Both substrate and product are measured, and the ratio of these values is used to generate % conversion of substrate to product by the LabChip EZ Reader. Typical reaction solutions contained 2% DMSO (±inhibitor), 2% glycerol, 10 mM MgCl₂, 1 mM DTT, 3.5 mM ATP, 0.005% Tween 20 (TW-20), 3 μM 5-FAM-Dyrktide, 4 nM (active sites) activated CDK6/Cyclin D3 in 40 mM HEPES buffer at pH 7.5.

Inhibitor Kᵢ determinations for activated CDK6/Cyclin D3 (LJIC-2009G1/2010+PO4) were initiated with the addition of ATP (50 μL final reaction volume), following an eighteen-minute pre-incubation of enzyme and inhibitor at 22° C. in the reaction mix. The reaction was stopped after 95 minutes by the addition of 50 μL of 30 mM EDTA. Kᵢ determinations were made from a plot of the fractional velocity as a function of inhibitor concentration fit to the Morrison equation with the enzyme concentration as a variable.

For CDK4 and CDK6 mobility shift assays, see also Morrison, J. F. (1969) Kinetics of the reversible inhibition of enzyme-catalysed reactions by tight-binding inhibitors, Biochimica et biophysica acta 185, 269-286; and Murphy, D. J. (2004) Determination of accurate KI values for tight-binding enzyme inhibitors: an in silico study of experimental error and assay design, Analytical biochemistry 327, 61-67.

### Biochemical Assays Results

Biological activity data for Compound 1 and PF-06873600 in the CDK2, CDK6 and CDK4 mobility shift assays are provided in Table 3 as Ki (nM).

TABLE 3
Biochemical potencies of Compounds 1 and 2 against
CDK2, CDK6 and CDK4 as measured in mobility-shift.

| | | Compound 1 | | Compound 2 (PF-06873600) | |
|---|---|---|---|---|---|
| CDK | cyclin | Ki values (nM) | n | Ki values (nM) | n |
| CDK2 | E1 | 0.08 | 33 | 0.11 | 6 |
| CDK4 | D1 | 0.79 | 33 | 1.2 | 16 |
| CDK6 | D3 | 3.1 | 39 | 2.5 | 15 |

Ki values (geometric mean, nM) and the 95% confidence interval (95% CI) with the number of independent replicates (n) are shown.

### Example 2: Cellular Pharmacology

Cellular CDK activity of Compound 1 and PF-06873600 were assessed in human CCNE1-amplified ovarian cancer cell line model OVCAR3 and human ER+ breast cancer cell line model MCF7 cells. OVCAR3 and MCF7 cells were treated for 1 h and 24 h respectively with a top dose of 10 μM of inhibitors diluted in DMSO and a 1:3 dilution dose curve to determine IC₅₀ values. OVCAR3 cells were treated overnight with 1 mM hydroxyurea to enrich for G1/S phase cells prior to treatment with inhibitors. Functional effects were measured in a 7-day anti-proliferation assay in one human ovarian cancer (OVCAR3), two human ER+ breast cancer (MCF7 and T47D) and three human non-small cell lung cancer (NCIH2087, NCIH358 and A549) cell line models. A 1:3 dilution dose curve in triplicates with a top dose of 10 μM of inhibitors in DMSO was used to determine IC₅₀ values.

### Cell Proliferation Assay

OVCAR3, MCF7, T47D, HCC1428, NCIH2087, NCIH358 or A549cells were seeded 1-3000 cells/well in 96-well plates in growth media containing 10% FBS and cultured overnight at 37° C. 5% CO₂. The following day, compounds were serially diluted from a 10 mM top dose for an 11-point 3-fold dilution curve in DMSO. Compound 1 and PF-06873600 were intermediately diluted 1:200 into growth media prior to diluting 1:5 on cells for final concentration 10 μM to 0.1 nM in 0.1% DMSO on cells. Cells were incubated at 37° C. 5% CO₂ for 7 days. CYQUANT Direct Cell Proliferation Assay (Molecular Probes, Eugene, OR) was then performed following manufacturer recommendations to determine the relative viable cell numbers on the Perkin Elmer Envision 2104 Multi Label Reader at 508 nM excitation and 527 nM emission wavelengths. IC₅₀ values were calculated by concentration-response curve fitting utilizing a four-parameter analytical method using GraphPad Prism software.

### Phospho-Serine 807/811 Rb ELISA

OVCAR3 or MCF7 cells were seeded at 25,000 cells/well in 100 μL growth media and allowed to adhere at 37° C. with 5% CO₂ overnight. OVCAR3 cells were treated overnight with 1 mM hydroxyurea to enrich for G1/S phase cells prior to treatment with inhibitors. The following day, compounds were serially diluted from a 10 mM top dose for an 11-point 3-fold dilution curve in DMSO. Compound 1 and PF-06873600 were intermediately diluted 1:200 into growth media prior to diluting 1:5 on cells for final concentration 10 μM to 0.1 nM in 0.1% DMSO on cells. OVCAR3 cells were treated for 1 hour, while MCF7cells were treated overnight, at 37° C. with 5% CO₂. Cells were lysed in 100 μL/well CST lysis buffer on ice and transferred to pre-coated and blocked anti-phospho-Ser807/811 Rb ELISA plates for overnight incubation at 4° C. Plates were washed to remove residual, unbound cellular proteins and total Rb detection antibody added for 90 minutes at 37° C. Following wash to remove unbound total Rb antibody, HRP tagged antibody was allowed to bind for 30 minutes at 37° C. Following wash to remove unbound HRP antibody, Glo Substrate Reagent was added and incubated protected from light for 5 to 10 minutes. Plates were read in luminescence mode and IC₅₀ values calculated.

### Results

### Compound 1 Inhibition of pRb in MCF7 ER+ Breast Cancer and OVCAR Ovarian Carcinoma Cells

The cellular activity of Compound 1 was evaluated in ER+, HER2− MCF7 cells and CCNE1-amplified ovarian carcinoma. Activity was determined by inhibition of phosphorylation of

Rb protein at serines 807/811 following treatment with Compound 1 or PF-06873600. Compound 1 demonstrated inhibition of cellular activity with a mean IC₅₀ of 147.8 nM while PF-06873600 had a mean IC₅₀ of 70.6 nM in the palbociclib-sensitive MCF7 breast cancer cellular model (Table 4). In OVCAR3 cells, Compound 1 treatment had a mean IC₅₀ of 16.2 nM, while PF-06873600 had a mean IC₅₀ of 21.8 nM (Error! Reference source not found.4).

### Compound 1 Inhibition of Proliferation of ER+ Human Breast Cancer Cells and Palbociclib Resistant Variants, Ovarian Carcinoma Cells, and NSCLC Cells

The effect of Compound 1 on cell proliferation was assessed in multiple ER+ breast cancer cell lines, including parental and palbociclib resistant cell lines.

Compound 1 treatment led to anti-proliferation IC₅₀ values ranging from 14.3 to 17.6 nM in MCF7, T47D, and HCC1428 parental ER+ breast cancer cell lines and 37.3 to 65.7 nM in palbociclib resistant (PalboR) derivatives of these cell lines (Error! Reference source not found.4). Compound 1 treatment of the CCNE1-amplified ovarian carcinoma cell line OVCAR3 was also effective at inhibiting cell proliferation with an IC₅₀ of 11.4 nM. Similar results were shown with NCIH2087, NCIH358, and A549 NSCLC cell lines (Error! Reference source not found.4).

The mean IC₅₀ values of CDK2/4/6 cellular activity suppression and anti-proliferation activity for Compound 1 and PF-06873600 are summarized in Table 4 below.

TABLE 4
Phospho-Rb Suppression and Anti-proliferation Activity of
Compound 1 and PF-06873600 in human cancer cell lines.

| | Compound 1 | | Compound 2 (PF-06873600) | |
|---|---|---|---|---|
| Cell Line | IC50 (nM) ± SEM | n | IC50 (nM) ± SEM | n |

Modulation of pRB Serine 807/811

| | | | | |
|---|---|---|---|---|
| MCF7 | 147.8 ± 3.1 | 6 | 70.6 ± 4.1 | 15 |
| OVCAR3 | 16.2 ± 1.3 | 6 | 21.8 ± 1.5 | 34 |

Proliferation Inhibition

| | | | | |
|---|---|---|---|---|
| MCF7 parental | 16.8 ± 6.7 | 12 | 38.6 ± 10.2 | 7 |
| MCF7 PalboR | 37.3 ± 2.8 | 3 | 111.2 ± 56.1 | 3 |
| T47D parental | 17.6 ± 2.5 | 6 | 70.6 ± 14.1 | 6 |
| T47D PalboR | 65.7 ± 8.8 | 4 | 253.2 ± 55.1 | 4 |
| HCC1428 parental | 14.3 ± 4.4 | 4 | 96.7 ± 26.4 | 4 |
| HCC1428 PalboR | 61.4 ± 4.9 | 4 | 268.6 ± 63.9 | 4 |
| OVCAR3 | 11.4 ± 4.2 | 5 | 70.0 ± 11.7 | 4 |
| NCIH2087 | 40.0 ± 6.8 | 2 | 79.8 | 1 |
| NCIH358 | 21.0 ± 2.2 | 2 | 39.0 | 1 |
| A549 | 121.3 ± 10.1 | 2 | 189.4 | 1 |

IC50 values (mean, nM) and standard error of the mean (SEM) with the number of independent replicates (n) are shown.

### Example 3: Prediction of Human Elimination Half-Life

This example demonstrates that Compound 1 exhibits a high blood to plasma ratio resulting in high blood binding and an unexpectly long predicted human elimination half-life.

Compound 1 has been evaluated according to methods well known in the art, for its ability to partition between human plasma and blood. Based on the observed partitioning and plasma protein binding, the blood binding was estimated based on human blood unbound fraction (fu_{b})=human plasma unbound fraction (fuₚ)/human blood-to-plasma partition ratio (BPR).

To determine and compare the predicted human elimination half-lives, compounds 1-4 were evaluated for their human blood-to-plasma partition ratio (BPR) along with other key in vitro ADME data including metabolic stability in human hepatocytes (CL, nt HHEP).

The predicted total clearance in human (CLb) was first determined based on the in vitro human metabolism data shown below using the equation:

CL_{b}=(Q·fu_{b}·CLᵢₙₜHHEP)/[(Q+fu_{b})·CLᵢₙₜHHEP]

(Scaling Clᵢₙₜ HHEP from μl/min/M to ml/min/kg based on hepatocellularity (120×10⁶/ g liver), g of liver per Kg bodyweight (21g/Kg), assuming hepatocyte binding is negligible and liver blood flow Q=20 ml/min/kg.)

The projected human volume of distribution at steady state in human blood (V_{dss_human}) was then calculated based on the animal (rat and/or dog) PK data. The V_{dss_human} was calculated factoring in differences in blood binding across compounds. Due to the high blood-to-plasma partition ratio in Compounds 1 and 4, the corresponding V_{dss_human} was limited to the blood volume with V_{dss_human} estimated to be 0.07 L/Kg.

Using the estimates of CL_{b} and V_{dss_human}, the predicted human elimination half-lives of compounds 1-4 were calculated according to the equation:

Half-life=0.693·V_{dss_human}/CL_{b}

Due to the unexpected much higher BPR and low CLᵢₙₜ HHEP for Compound 1, a significantly longer human half-life was predicted (i.e., predicted t_{1/2}=74 hours for Compound 1 vs. predicted t_{1/2}=6 hours for PF-06873600). However, because the observed human t_{1/2} for PF-06873600 is 3 hours (˜2 fold over prediction), the predicted t_{1/2} for Compounds 1, 3 and 4 were adjusted accordingly. The adjusted predicted t_{1/2} of Compound 1 is 34 hours which is significantly higher than the observed t_{1/2} of PF-06873600 and the adjusted predicted t_{1/2} of Compounds 3 and 4. This longer half-life of Compound 1 may provide for QD or less frequent dosing with a minimized Cₘₐₓ concentration.

TABLE 5

| | | Compound 2 | | |
|---|---|---|---|---|
| | Compound 1 | (PF-06873600) | Compound 3 | Compound 4 |
| CLᵢₙₜ HHEP: CLᵢₙₜ in human | ~2 | ~2 | ~4 | ~3 |
| hepatocytes (μl/min/M) (in vitro) | | | | |
| fuₚ: human plasma unbound | 0.174 | 0.288 | 0.297 | 0.148 |
| fraction | | | | |
| BPR: human blood-to-plasma | 79 | 0.9 | 0.9 | 28 |
| partition ratio | | | | |
| fu_{b}: human blood unbound | 0.0022 | 0.32 | 0.33 | 0.00529 |
| fraction | | | | |
| CL_{b}: predicted total clearance | 0.011 | 1.5 | 2.8 | 0.040 |
| (ml/min/kg) | | | | |
| Rat Vdss (L/Kg) | 1.5 | Use dog PK | Use dog PK | 0.43 |
| Dog Vdss (L/Kg) | 1.5 | 1.1 | 0.78 | ND |
| Rat V_{dss unbound}:_ (Rat plasma | 10.8 | Use dog PK | Use dog PK | 8.8 |
| V_{dss}/rat plasma fu) (L/Kg) | | | | |
| Dog V_{dss unbound}: (Dog V_{dss}/dog | 6.7 | 2.8 | 2.9 | ND |
| plasma fu) (L/Kg) | | | | |
| Mean V_{dss unbound}: (mean animal | 8.8 | 2.8 | 2.9 | 8.8 |
| V_{dss unbound} values) (L/Kg) | | | | |
| V_{dss}_{_{—}}ₕᵤₘₐₙ*: projected volume of | 0.019 L/kg | 0.9 L/Kg | 1.0 L/Kg | 0.047 L/Kg |
| distribution (steady state) | (not used); | | | (not used); |
| | Limited to | | | Limited to |
| | 0.07 L/kg | | | 0.07 L/Kg |
| predicted t_{1/2}: predicted human | 72 | 7 | 4 | 21 |
| elimination half-life (hour) | | | | |
| (adjusted) predicted t_{1/2} based | 34 | ~3 | 3 | 10 |
| on PF-06873600's observed t_{1/2} | | (observed t_{1/2}) | | |

*V_{dss}_{_{—}}ₕᵤₘₐₙ = (Mean V_{dss unbound}) · (fu_{b}); Lower limit of V_{dss}_{_{—}}ₕᵤₘₐₙ = 0.07 L/kg

### Human hepatocyte stability

Test compound (i.e., compound 1, 2, 3, or 4) (1 μM) was incubated with human hepatocytes at 0.5 million cells/mL at 37° C. in an incubator (relative humidity≥90%, 5% CO₂/air) for 4 hours. At various time points, samples were taken and analyzed by LC-MS/MS. CLᵢₙₜ HHEP was calculated based on loss of parent compound over time using equations discussed in “A novel relay method for determining low-clearance values,” Drug Metab Dispos., 2012; 40(9):1860-5.

### Human Plasma Protein Binding

Frozen plasma in K3EDTA was purchased from BiolVT and Dulbecco's phosphate buffered saline (DPBS) and HCl were purchased from Sigma.

Human plasma unbound fraction (fu r) was determined by equilibrium dialysis using an HTD 96 device (HTDialysis, LLC, Gales Ferry, CT) assembled with 12-14k MWCO membranes. Plasma was thawed and adjusted to pH 7.4 with 1 N HCl prior to use. Dialysis chambers were loaded with 150 μL plasma and 150 μL PBS in the donor and receiver chambers, respectively. The dialysis plate was sealed with a gas-permeable membrane and stored in a 37° C. water-jacked incubator maintained at 75% relative humidity and 5% CO₂, on a 100 rpm plate shaker. After a 6-hour incubation, samples were matrix-matched and quench by protein precipitation, followed by LC-MS analysis. A set of satellite samples was included to measure stability after a 6-hour incubation. Incubations were conducted with 4 to 12 replicates. The fuₚ was calculated by dividing the analyte-to-internal standard peak area ratio or analyte concentration in the buffer sample by the signal in the donor sample, corrected for any dilution factors. All incubations had >70% analyte recovery and >70% stability in 6 hours.

### Human Blood-to-Plasma Partition Ratio (BPR)

The distribution of compounds between plasma and whole blood was determined by adding the test compound (i.e., compounds 1, 2, 3, or 4) (1 mM) in fresh blood and incubating at 37° C. for 1 hour in an incubator (90% humidity, 5% CO₂/air) on a shaker (450 rpm) according to methods described in Novak, J.J. et al, “Effects of low temperature on blood-to-plasma ratio measurement,” Biopharm Drug Dispos. 2021, 42(5):234-41. At the end of the incubations, blood and plasma samples were matrix-matched and analyzed by LC-MS/MS. BPR was calculated by dividing the blood and plasma measure peak areas.

### Human Blood Binding

Human blood unbound fraction (fu_{b}) was calculated by dividing fuₚ by BPR.

### Preclinical Animal Pharmacokinetic Studies

All activities involving animals were carried out in accordance with federal, state, local and institutional guidelines governing the use of laboratory animals in research in an Accreditation of Laboratory Animal Care (AAALAC) accredited facility and were reviewed and approved by Pfizer's Institutional Animal Care and Use Committee.

Rat PK studies were done at Pfizer (Groton, CT) or BioDuro Pharmaceutical Product Development Inc. (Shanghai, PRC); Jugular vein-cannulated male Wistar-Hannover rats were purchased from Charles River Laboratories, Inc. (Wilmington, MA) or Vital River (Beijing, China) and were typically 7-10 weeks of age at the time of dosing. During the pharmacokinetic studies all animals were housed individually. Access to food and water was provided ad libitum.

Compounds were administered i.v. via the tail vein (n=2 or 3), dosed as a 1 mg/ml solution using standard compatible excipients at 1 mL/kg for a resulting dose of 2 mg/kg. Serial blood samples were collected via the jugular vein cannula at predetermined timepoints after dosing. Animals were monitored for pain or distress throughout the study, with at least daily monitoring during normal husbandry prior to study start. At the completion of the study, animals were euthanized by overdose of inhaled anesthesia followed by exsanguination. Blood samples were collected into tubes containing K3EDTA and stored on ice until centrifugation to obtain plasma, which was stored frozen at −20° C. or lower. Urine samples were collected at room temperature and stored frozen at 20° C. or lower at the end of each time interval.

Dog PK studies were done at Pfizer (Groton, CT); animal care and in vivo procedures were conducted according to guidelines from the Pfizer Institutional Animal Care and Use Committee. Male Beagle dogs were purchased from Marshall BioResources (North Rose, New York) and were typically 1-5 years of age at the time of dosing.

Compounds were administered i.v. via the cephalic vein (n=2), dosed as a solution using standard compatible excipients at 0.5 mL/kg for a resulting dose of 0.1 mg/kg. Serial blood samples were collected via the jugular vein at predetermined timepoints after dosing. Animals were monitored for pain or distress throughout the study, with at least daily monitoring during normal husbandry prior to study start. Blood samples were collected into tubes containing K3EDTA and stored on ice until centrifugation to obtain plasma, which was stored frozen at −20° C. or lower. Urine samples were collected at room temperature and stored frozen at −20° C. or lower at the end of each time interval.

Plasma samples from the animal PK studies were processed using protein precipitation with acetonitrile: methanol (1:1) containing internal standard verapamil followed by quantitation by LC-MS/MS against a standard curve prepared in blank plasma. Pharmacokinetic parameters were calculated using noncompartmental analysis (Watson v.7.5, Thermo Scientific).

### Example 4: Mouse Early Toxicology Screening (ETS) Study

Three groups of ten-week-old male CD1 mice were separately evaluated in early toxicology screening studies. Group 1 (n=5/group) were administered an oral dose of 25, 50 and 75 mg/kg BID of Compound 1 for 14 days. Group 2 (n=5/group) were administered an oral dose of 25, 75 and 150 mg/kg BID of PF-06873600 for 14 days. A vehicle control Group 3 (n=5/group) were also included where vehicle was administered BID for 14 days. Study evaluations included clinical observations, body weights, clinical pathology, toxicokinetics, gross pathology and microscopic pathology. No neutropenia was observed in all dosing levels in Group 1 (i.e., treatment with Compound 1).

These results were unexpected because, as mentioned in the Background, previous studies have shown that treatment with CDK4/6 inhibitors, which inhibits CDK6, can lead to hematologic adverse events, but here, while Compound 1 exhibits CDK6 activity as shown in the biochemical assay (see Table 3), no neutropenia was observed in Group 1.

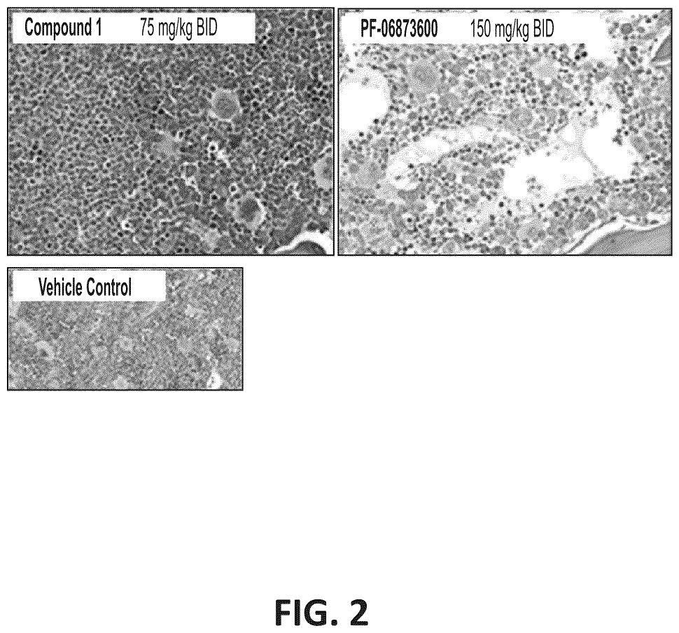

At the end of the ETS study on day 14, mice were sacrificed and images of the mouse bone marrow from each study group (n=5) were obtained. FIG. 2 shows the histology images of the mouse bone marrow biopsy from (1) Group 1 at 75 mg/kg BID Compound 1, (2) Group 2 at 150 mg/kg BID PF-06873600, and (3) Group 3 with vehicle control. The imaging assessment of the mouse bone marrow provides confirmation of the lack of impact on the mouse bone marrow with Compound 1. The vehicle and group 1 images appear very similar with densly healthy populated cells where in contrast PF-06873600 demonstrates vacuoles and large spaces between cells in the bone marrow image indicating cell death/apoptosis.

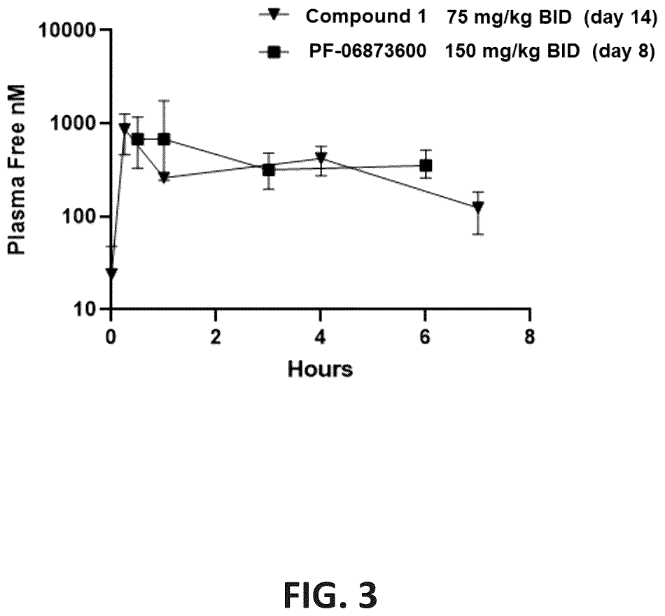

The systemic exposure of Compound 1 and PF-06873600 were also compared. Exposures from mouse toxicity studies were determined using plasma samples collected as timepoints on day 8 or 14 of the study and were processed using protein precipitation with solvent mixture acetonitrile:methanol (1:1) containing internal standard verapamil followed by quantitation by LC-MS/MS against a standard curve prepared in blank plasma. The free plasma concentrations were calculated using the mouse plasma protein binding for compound 1 (fuₚ=0.182) and PF-06873600 (fuₚ=0.277). (FIG. 3)

Surprisingly, while both Compound 1 and PF-06873600 exhibit CDK6 activity and achieve similar free systemic exposure in the mouse toxicology study (BID oral dosing over 14 days), no neutropenia was observed in Group 1 receiving treatment with Compound 1. These observed differences between the treatment Group 1 and Group 2 are not currently understood. However, as demonstrated above, the predicted (adjusted) human half-life of Compound 1 is much longer than that of the observed human half-life of PF-06873600, and Compound 1 has shown reduced impact on circulating neutrophils, this would provide an improved profile for Compound 1 as compared to PF-06873600.

It will be apparent to those skilled in the art that various modifications and variations may be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

We claim:

1. A compound of Formula (I) 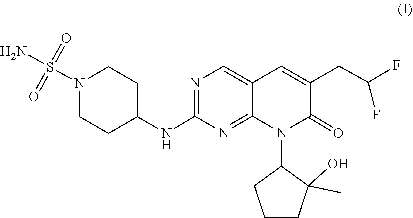 [SMILES: CC1(O)CCCC1n1c(=O)c(CC(F)F)cc2cnc(NC3CCN(S(N)(=O)=O)CC3)nc21]
or a pharmaceutically acceptable salt thereof.

2. The compound of claim 1, or pharmaceutically acceptable salt thereof, wherein the compound of Formula (I) has the absolute stereochemistry as shown in Formula (I-A), (I-B), (I-C), or (I-D):

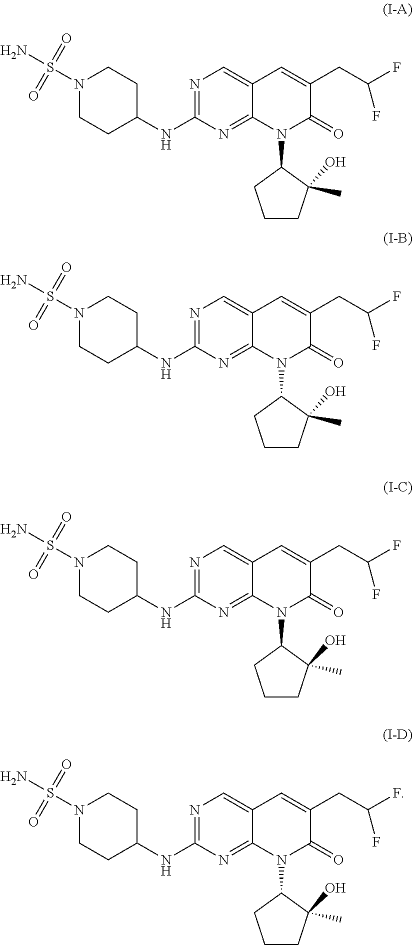 [SMILES: C[C@@]1(O)CCC[C@H]1n1c(=O)c(CC(F)F)cc2cnc(NC3CCN(S(N)(=O)=O)CC3)nc21 ; C[C@@]1(O)CCC[C@@H]1n1c(=O)c(CC(F)F)cc2cnc(NC3CCN(S(N)(=O)=O)CC3)nc21 ; C[C@]1(O)CCC[C@H]1n1c(=O)c(CC(F)F)cc2cnc(NC3CCN(S(N)(=O)=O)CC3)nc21 ; C[C@]1(O)CCC[C@@H]1n1c(=O)c(CC(F)F)cc2cnc(NC3CCN(S(N)(=O)=O)CC3)nc21]

3. The compound of claim 1 which is 4-((6-(2,2-difluoroethyl)-8-((1R,2R)-2-hydroxy-2-methylcyclopentyl)-7-oxo-7,8-dihydropyrido[2,3-d]pyrimidin-2- yl)amino)piperidine-1-sulfonamide of Formula (I-A) or a pharmaceutically acceptable salt thereof.

4. The compound of claim 1 which is 4-((6-(2,2-difluoroethyl)-8-((1S,2R)-2-hydroxy-2-methylcyclopentyl)-7-oxo-7,8-dihydropyrido[2,3-d]pyrimidin-2- yl)amino)piperidine-1-sulfonamide of Formula (I-B) or a pharmaceutically acceptable salt thereof.

5. The compound of claim 1 which is 4-((6-(2,2-difluoroethyl)-8-((1R,2S)-2-hydroxy-2-methylcyclopentyl)-7-oxo-7,8-dihydropyrido[2,3-d]pyrimidin-2- yl)amino)piperidine-1-sulfonamide of Formula (I-C) or a pharmaceutically acceptable salt thereof.

6. The compound of claim 1 which is 4-((6-(2,2-difluoroethyl)-8-((1S,2S)-2-hydroxy-2-methylcyclopentyl)-7-oxo-7,8-dihydropyrido[2,3-d]pyrimidin-2- yl)amino)piperidine-1-sulfonamide of Formula (I-D) or a pharmaceutically acceptable salt thereof.

7. A compound which is 4-((6-(2,2-difluoroethyl)-8-((1R,2R)-2-hydroxy-2-methylcyclopentyl)-7-oxo-7,8-dihydropyrido[2,3-d]pyrimidin-2- yl)amino)piperidine-1-sulfonamide.

8. The compound of claim 7 having the chemical structure

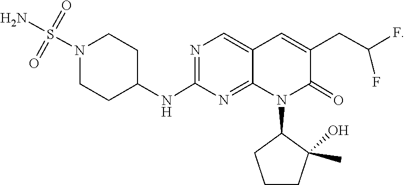 [SMILES: C[C@@]1(O)CCC[C@H]1n1c(=O)c(CC(F)F)cc2cnc(NC3CCN(S(N)(=O)=O)CC3)nc21]

9. A pharmaceutically acceptable salt of 4-((6-(2,2-difluoroethyl)-8-((1R,2R)-2-hydroxy-2-methylcyclopentyl)-7-oxo-7,8-dihydropyrido[2,3-d]pyrimidin-2-yl)amino)piperidine-1-sulfonamide.

10. An anhydrous crystalline form of 4-((6-(2,2-difluoroethyl)-8-((1R,2R)-2-hydroxy-2-methylcyclopentyl)-7-oxo-7,8-dihydropyrido[2,3-d]pyrimidin-2- yl)amino)piperidine-1-sulfonamide, free base (Form 1) having a powder X-ray diffraction (PXRD) pattern comprising peaks at 2θ values of: 4.8, 14.3 and 19.7 °2θ±0.2 °2θ.

11. The anhydrous crystalline of claim 10, having a PXRD pattern further comprising a peak at a 20 value of: 10.6 °2θ±0.2 °2θ.

12. The anhydrous crystalline of claim 11, having a PXRD pattern further comprising a peak at a 2θ value of: 19.1 °2θ±0.2 °2θ.

13. The anhydrous crystalline of claim 10, having a PXRD pattern comprising peaks at 2θ values essentially the same as shown in FIG. 1.

14. The anhydrous crystalline of claim 10, having a powder X-ray diffraction (PXRD) pattern comprising three, four, five, or more than five peaks in Table 1 in °2θ±0.2 °2θ.

15. The anhydrous crystalline of claim 10 wherein the crystalline form is substantially pure.

16. A pharmaceutical composition comprising a compound according to any one of claims 1 to 15 or a pharmaceutically acceptable salt thereof, and at least one pharmaceutically acceptable excipient.

17. A method for treating cancer, comprising administering to a subject in need thereof a therapeutically effective amount of the compound of any one of claims 1 to 16, or a pharmaceutically acceptable salt thereof.

18. The method for treating cancer of claim 17, further comprising administering an additional anticancer therapeutic agent.

19. The method for treating cancer of claim 17, wherein the cancer is selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer and thyroid cancer.

20. The method for treating cancer of claim 17, wherein the cancer is breast cancer or ovarian cancer.

21. The method for treating cancer of claim 20, wherein the breast cancer or ovarian cancer is characterized by amplification or overexpression of cyclin E1 (CCNE1) or cyclin E2 (CCNE2).

22. The method for treating cancer of claim 20, wherein the breast cancer is ER-positive/HR-positive breast cancer, HER2-negative breast cancer, ER-positive/HR-positive breast cancer, HER2-positive breast cancer, triple negative breast cancer (TNBC), or inflammatory breast cancer.

23. The method of treating cancer of claim 20, wherein the breast cancer is advanced or metastatic breast cancer.

24. A pharmaceutical combination comprising a compound according to any one of claims 1 10 to 15 or a pharmaceutically acceptable salt, at least one additional therapeutic agent, and at least one pharmaceutically acceptable excipient.
